SEQUENCE LISTING (18 sequences)

SEQ ID NO: 1 - protein, 26 aa, Unknown
  Description of Unknown Signal peptide from Kaposi fibroblast growth factor
AAVALLPAVLLALLAPVNRKRNKLMP

SEQ ID NO: 2 - protein, 11 aa, Human immunodeficiency virus
YGRKKRRQRRR

SEQ ID NO: 3 - protein, 15 aa, Homo sapiens
VTVLALGALAGVGVG

SEQ ID NO: 4 - protein, 12 aa, Artificial Sequence
  Description of Artificial Sequence Synthetic peptide
AAVLLPVLLAAP

SEQ ID NO: 5 - protein, 5 aa, Artificial Sequence
  Description of Artificial Sequence Synthetic peptide
YVADX

SEQ ID NO: 6 - protein, 5 aa, Artificial Sequence
  Description of Artificial Sequence Synthetic peptide
DXXDX

SEQ ID NO: 7 - protein, 9 aa, Artificial Sequence
  Description of Artificial Sequence Synthetic peptide
RXXXXXXRX

SEQ ID NO: 8 - protein, 9 aa, Artificial Sequence
  Description of Artificial Sequence Synthetic peptide
KXXXXXXRX

SEQ ID NO: 9 - protein, 6 aa, Artificial Sequence
  Description of Artificial Sequence Synthetic peptide
XRTKRX

SEQ ID NO: 10 - protein, 5 aa, Artificial Sequence
  Description of Artificial Sequence Synthetic peptide
RVRRX

SEQ ID NO: 11 - protein, 5 aa, Artificial Sequence
  Description of Artificial Sequence Synthetic peptide
RVRRX

SEQ ID NO: 12 - protein, 6 aa, Artificial Sequence
  Description of Artificial Sequence Synthetic peptide
PXWVPX

SEQ ID NO: 13 - protein, 4 aa, Artificial Sequence
  Description of Artificial Sequence Synthetic peptide
WVAX

SEQ ID NO: 14 - protein, 4 aa, Artificial Sequence
  Description of Artificial Sequence Synthetic peptide
XFXX

SEQ ID NO: 15 - protein, 4 aa, Artificial Sequence
  Description of Artificial Sequence Synthetic peptide
XYXX

SEQ ID NO: 16 - protein, 4 aa, Artificial Sequence
  Description of Artificial Sequence Synthetic peptide
XWXX

SEQ ID NO: 17 - protein, 12 aa, Artificial Sequence
  Description of Artificial Sequence Synthetic peptide
DRYIPFHLLVYS

SEQ ID NO: 18 - protein, 32 aa, Artificial Sequence
  Description of Artificial Sequence Synthetic polypeptide
CSNLSTCVLGKLSQELHKLQTYPRTXXGXXTX

(19) United States
(12) Patent Application Publication
(10) Pub. No.: US 2015/0118315 A1
(43) Pub. Date: Apr. 30, 2015

(54) AROMATIC-CATIONIC PEPTIDES AND USES OF SAME

(71) Applicant: D. Travis Wilson, Newton, MA (US)

(72) Inventor: D. Travis Wilson, Newton, MA (US)

(73) Assignee: Stealth Peptides International, Inc., Monaco (MC)

(21) Appl. No.: 14/125,826

(22) PCT Filed: Jun. 13, 2012
(86) PCT No.: PCT/US12/42261

§ 371 Date: Jun. 19, 2014

Related U.S. Application Data
(60) Provisional application No. 61/496,994, filed on Jun. 14, 2011.
(60) Provisional application No. 61/505,479, filed on Jul. 7, 2011.

(51) Int. Cl. A61K 47/28; A61K 38/07; A61K 47/24; A61K 38/16; A61K 47/12; A61K 47/18; A61K 38/08; A61K 38/10

(52) U.S. Cl. CPC A61K 47/28; A61K 38/08; A61K 38/07; A61K 38/10; A61K 38/16; A61K 47/12; A61K 47/186; A61K 47/24
USPC 424/490; 514/21.9; 514/21.8; 514/21.7; 514/21.6; 514/21.5; 514/21.4; 514/21.3

(57) ABSTRACT

A finished pharmaceutical product adapted for oral delivery of an aromatic-cationic peptide, wherein the product comprises a therapeutically effective amount of the peptide; at least one pharmaceutically acceptable pH-lowering agent; and at least one absorption enhancer effective to promote bioavailability of the active agent. The product is adapted for use in a method for enhancing the bioavailability of a therapeutic aromatic-cationic peptide delivered orally.

### CROSS REFERENCE TO RELATED APPLICATIONS

This application claims priority to U.S. Provisional Application Ser. No. 61/496,994, filed Jun. 14, 2011, and U.S. Provisional Application Ser. No. 61/505,479, filed: Jul. 7, 2011, both of which are hereby incorporated by reference in their entirety.

### FIELD

The present technology relates to aromatic-cationic peptide pharmaceuticals where the active compounds include a plurality of amino acids and at least one peptide bond in their molecular structures, and to methods of quickly providing good bioavailability of such peptide active compounds when administered to subjects.

### BACKGROUND

Peptide pharmaceuticals used in the prior art frequently have been administered by injection or by nasal administration. However, injection and nasal administration are significantly less convenient, and involve more patient discomfort than, for example, oral administration.

Often this inconvenience or discomfort results in substantial patient noncompliance with a treatment regimen. Oral administration tends to be problematic, however, because peptide active compounds are very susceptible to degradation in the stomach and intestines. Thus, there is a need in the art for more effective and reproducible oral administration of peptide pharmaceuticals like insulin, salmon calcitonin and others discussed in more detail herein.

Proteolytic enzymes of both the stomach and intestines may degrade peptides, rendering them inactive before they can be absorbed into the bloodstream. Any amount of peptide that survives proteolytic degradation by proteases of the stomach (typically having acidic pH optima) is later confronted with proteases of the small intestine and enzymes secreted by the pancreas (typically having neutral to basic pH optima).

Specific difficulties arising from the oral administration of peptides involve the size of the molecule, and the charge distribution it carries. These physical properties may make it more difficult for the peptide to penetrate the mucus along intestinal walls or to cross the intestinal brush border membrane into the blood, and may result in limited bioavailability.

Oral dosage forms which at least partially surmount many of the difficulties described above are disclosed and claimed in U.S. Pat. Nos. 5,912,014 and 6,086,918 to Stern et al., issued Jun. 15, 1999 and Jul. 11, 2000, respectively, which are incorporated herein by reference. Both patents describe peptide dosage formulations which target release of the peptide to the intestine and which enhance bioavailability by administering the peptide in an oral dosage formulation which comprises, in addition to the peptide, at least one pharmaceutically acceptable pH-lowering agent and at least one absorption enhancer effective to promote bioavailability of the peptide. The dosage formulation is, moreover, coated with an enteric coating capable of conducting the peptide, the absorption enhancer and the pH-lowering agent through a subject's stomach, while protecting the peptide from degradation by stomach proteases. Thereafter, the coating dissolves and the peptide, absorption enhancer and pH lowering agent are released together into the intestine of the subject.

In certain instances, however, the condition to be treated by the oral peptide would benefit from more rapid remediation than that provided by the relatively slow dissolution of an enteric coating and related release of the active component(s) within the intestine. One particular example of a condition which benefits from such rapid remediation involves the area of pain relief, where the speed with which such relief is achieved is obviously an important, if not critical, factor to a patient. Furthermore, it is not always required that the aromatic-cationic peptide be transported all of the way through the stomach and into the intestine. That is, in the case of certain aromatic-cationic peptides, including but not limited to various pain-relievers, it may be most efficacious for absorption of the therapeutic peptide is thought to occur prior to entry of the formulation into the intestine, e.g., as the material passes down the esophagus or when it is within the patient's stomach. Under such circumstances, while oral bioavailability is still a factor to be considered, patients and/or clinicians may be willing to accept a limited reduction in bioavailability if such reduction is balanced by a corresponding increase in the speed of absorption, and thus of action, by the therapeutic peptide(s) contained within the formulation.

There has thus been a long-felt need for an oral peptide formulation which is capable of more rapid therapeutic action, i.e., in contrast to the formulations described in the '014 and '918 patents discussed above, while still providing a desirable degree of bioavailability.

Normally, the plasma membrane of eukaryotic cells is impermeable to large peptides or proteins. However, certain hydrophobic amino acid sequences, variously called as ferry peptides or membrane translocating sequences, when fused to the N- or C-terminus of functional proteins, can act as membrane translocators, and mediate the transport of these proteins into living cells. This method of protein delivery into cells, while potentially very useful, has two main drawbacks. First, the protein cannot be targeted to any specific cell type. Therefore, once it is injected and enters the circulation, it will presumably enter all cell types in a non-specific, non-receptor mediated manner. This would cause a huge dilution effect, such that very high concentrations of the protein need to be injected in order to achieve an effective concentration in the target cell type. Also, the protein could be extremely toxic when it enters cells in non-target tissues. A third drawback is that the continued presence of the ferry peptide could make the protein very antigenic, and could also interfere with its biological activity. These above drawbacks would apply whether the fusion was delivered by injection or nasal or oral route.

Nasal delivery is also frequently plagued by low bioavailability of the therapeutic peptide. Even where nasal delivery is possible, manufacturing costs can be undesirably high because of the large concentration of therapeutic peptide required to provide clinical efficacy in view of low bioavailability occasioned by the difficulty of peptides crossing the nasal mucosa.

Therapeutic peptides are often poorly absorbed by tissues, and are readily degraded by bodily fluids. For this reason, formulations were developed for the administration of peptide therapeutics via the nasal route. The nasal formulation was designed to be stored in a multi-dose container that was stable for an extended period of time and resisted bacterial contamination. The preservative in the formulation, benzalkonium chloride, was found to enhance the absorption of the peptide therapeutic. However, benzalkonium chloride was reported (P. Graf et al., Clin. Exp. Allergy 25:395-400; 1995) to aggravate rhinitis medicamentosa in healthy volunteers who were given a decongestant nasal spray containing the preservative. It also had an adverse effect on nasal mucosa (H. Hallen et al., Clin. Exp. Allergy 25:401-405; 1995), Berg et al. (Laryngoscope 104:1153-1158; 1994) disclose that respiratory mucosal tissue that was exposed in vitro underwent severe morphological alterations. Benzalkonium chloride also caused significant slowing of the mucocilary transport velocity in the ex vivo frog palate test (P. C. Braga et al., J. Pharm. Pharmacol. 44:938-940; 1992).

### SUMMARY

The present technology relates to pharmaceutical formulations for the delivery of aromatic-cationic peptides or a pharmaceutically acceptable salt thereof, such as acetate salt or trifluoroacetate salt. In one aspect, the present technology relates to a finished pharmaceutical product adapted for oral delivery of an aromatic-cationic peptide, the product comprising: (a) a therapeutically effective amount of the active peptide; (b) at least one pharmaceutically acceptable pH-lowering agent; and (c) at least one absorption enhancer effective to promote bioavailability of the active agent, wherein the pH-lowering agent is present in the finished pharmaceutical product in a quantity which, if the product were added to 10 milliliters of 0.1M aqueous sodium bicarbonate solution, would be sufficient to lower the pH of the solution to no higher than 5.5, and wherein an outer surface of the product is substantially free of an acid-resistant protective vehicle.

In some embodiments, the pH-lowering agent is present in a quantity which, if the product were added to 10 milliliters of 0.1M sodium bicarbonate solution, would be sufficient to lower the pH of the solution to no higher than 3.5. In some embodiments, the absorption enhancer is an absorbable or biodegradable surface active agent. In some embodiments, the surface active agent is selected from the group consisting of acylcarnitines, phospholipids, bile acids and sucrose esters. In some embodiments, the absorption enhancer is a surface active agent selected from the group consisting of: (a) an anionic agent that is a cholesterol derivative, (b) a mixture of a negative charge neutralizer and an anionic surface active agent, (c) non-ionic surface active agents, and (d) cationic surface active agents.

In some embodiments, the finished pharmaceutical product further comprises an amount of a second peptide that is not a physiologically active peptide effective to enhance bioavailability of the aromatic-cationic peptide. In some embodiments, the finished pharmaceutical product comprises at least one pH-lowering agent with a solubility in water of at least 30 grams per 100 milliliters of water at room temperature. In some embodiments, the finished pharmaceutical product comprises granules containing a pharmaceutical binder and, uniformly dispersed in the binder, the pH-lowering agent, the absorption enhancer and the aromatic-cationic peptide.

In some embodiments, the finished pharmaceutical product comprises a lamination having a first layer comprising the at least one pharmaceutically acceptable pH-lowering agent and a second layer comprising the therapeutically effective amount of the active peptide; the product further comprising the at least one absorption enhancer effective to promote bioavailability of the active agent, wherein the first and second layers are united with each other, but the at least one pH-lowering agent and the peptide are substantially separated within the lamination such that less than about 0.1% of the peptide contacts the pH-lowering agent to prevent substantial mixing between the first layer material and the second layer material and thus to avoid interaction in the lamination between the pH-lowering agent and the peptide.

In some embodiments, the finished pharmaceutical product comprises a pH-lowering agent selected from the group consisting of citric acid, tartaric acid and an acid salt of an amino acid. In some embodiments, the pH-lowering agent is selected from the group consisting of dicarboxylic acids and tricarboxylic acids. In some embodiments, the pH-lowering agent is present in an amount not less than 300 milligrams.

In some embodiments, the finished pharmaceutical product comprises an aromatic-cationic peptide or a pharmaceutically acceptable salt thereof, such as acetate salt or trifluoroacetate salt. In some embodiments, the aromatic-cationic peptide comprises the amino acid sequence Phe-D-Arg-Phe-Lys-NH₂ or a pharmaceutically acceptable salt thereof, such as acetate salt or trifluoroacetate salt. In some embodiments, the aromatic-cationic peptide comprises the amino acid sequence D-Arg-2′6′-Dmt-Lys-Phe-NH₂ or a pharmaceutically acceptable salt thereof, such as acetate salt or trifluoroacetate salt. In some embodiments, the aromatic-cationic peptide is selected from the group consisting of:

Phe-Arg-D-His-Asp
Met-Tyr-D-Lys-Phe-Arg
Phe-D-Arg-His
Tyr-D-Arg-Phe-Lys-NH₂
2′6′-Dmt-D-Arg-Phe-Lys-NH₂
2′6′-Dmt-D-Arg-Phe Orn-NH₂
2′6′-Dmt-D-Cit-Phe Lys-NH₂
Phe-D-Arg-2′6′-Dmt-Lys-NH₂
2′6′-Dmt-D-Arg-Phe-Ahp-NH₂
H-Phe-D-Arg-Phe-Lys-Cys-NH₂
2′6′-Dmp-D-Arg-2′6′-Dmt-Lys-NH₂
2′6′-Dmp-D-Arg-Phe-Lys-NH₂
Tyr-Arg-Phe-Lys-Glu-His-Trp-D-Arg
Lys-Gln-Tyr-D-Arg-Phe-Trp
D-Arg-2′6′-Dmt-Lys-Trp-NH₂
D-Arg-Trp-Lys-Trp-NH₂
D-Arg-2′6′-Dmt-Lys-Phe-Met-NH₂
D-Arg-2′6′-Dmt-Lys(N^{α}Me)-Phe-NH₂
D-Arg-2′6′-Dmt-Lys-Phe(NMe)—NH₂
D-Arg-2′6′-Dmt-Lys(N^{α}Me)-Phe(NMe)—NH₂
D-Arg(N^{α}Me)-2′6′-Dmt(NMe)-Lys(N^{α}Me)-Phe(NMe)—NH₂
D-Arg-2′6′-Dmt-Lys-Phe-Lys-Trp-NH₂
D-Arg-2′6′-Dmt-Lys-2′6′-Dmt-Lys-Trp-NH₂
D-Arg-2′6′-Dmt-Lys-Phe-Lys-Met-NH₂
D-Arg-2′6′-Dmt-Lys-2′6′-Dmt-Lys-Met-NH₂
D-Arg-2′6′-Dmt-Lys-Phe-Sar-Gly-Cys-NH₂
D-Arg-Ψ[CH₂—NH]2′6′-Dmt-Lys-Phe-NH₂
D-Arg-2′6′-Dmt-Ψ[CH₂—NH]Lys-Phe-NH₂
D-Arg-2′6′-Dmt-LysΨ[CH₂—NH]Phe-NH₂
D-Arg-2′6′-Dmt-Ψ[CH₂—NH]Lys-Ψ[CH₂—NH]Phe-NH₂
Lys-D-Arg-Tyr-NH₂
D-Tyr-Trp-Lys-NH₂
Trp-D-Lys-Tyr-Arg-NH₂
Tyr-His-D-Gly-Met
Tyr-D-Arg-Phe-Lys-Glu-NH₂
Met-Tyr-D-Arg-Phe-Arg-NH₂
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr-D-Phe-Glu-D-Asp-D-Asp-D-His-D-Lys-Arg-Trp-NH₂
Ala-D-Phe-D-Arg-Tyr-Lys-D-Trp-His-D-Tyr-Gly-Phe
Tyr-D-His-Phe-D-Arg-Asp-Lys-D-Arg-His-Trp-D-His-Phe
Phe-Phe-D-Tyr-Arg-Glu-Asp-D-Lys-Arg-D-Arg-His-Phe-NH₂
Phe-Tyr-Lys-D-Arg-Trp-His-D-Lys-D-Lys-Glu-Arg-D-Tyr-Thr
Tyr-Asp-D-Lys-Tyr-Phe-D-Lys-D-Arg-Phe-Pro-D-Tyr-His-Lys
Glu-Arg-D-Lys-Tyr-D-Val-Phe-D-His-Trp-Arg-D-Gly-Tyr-Arg-D-Met-NH₂
Arg-D-Leu-D-Tyr-Phe-Lys-Glu-D-Lys-Arg-D-Trp-Lys-D-Phe-Tyr-D-Arg-Gly
D-Glu-Asp-Lys-D-Arg-D-His-Phe-Phe-D-Val-Tyr-Arg-Tyr-D-Tyr-Arg-His-Phe-NH₂
Asp-Arg-D-Phe-Cys-Phe-D-Arg-D-Lys-Tyr-Arg-D-Tyr-Trp-D-His-Tyr-D-Phe-Lys-Phe
His-Tyr-D-Arg-Trp-Lys-Phe-D-Asp-Ala-Arg-Cys-D-Tyr-His-Phe-D-Lys-Tyr-His-Ser-NH₂
Gly-Ala-Lys-Phe-D-Lys-Glu-Arg-Tyr-His-D-Arg-D-Arg-Asp-Tyr-Trp-D-His-Trp-His-D-Lys-Asp
Thr-Tyr-Arg-D-Lys-Trp-Tyr-Glu-Asp-D-Lys-D-Arg-His-Phe-D-Tyr-Gly-Val-Ile-D-His-Arg-
Tyr-Lys-NH₂

In one aspect, the present technology provides methods for enhancing the bioavailability of a therapeutic aromatic-cationic peptide delivered orally in a subject in need of such enhancement, the method comprising selectively releasing the aromatic-cationic peptide, together with at least one pH-lowering agent and at least one absorption enhancer, into the subject's alimentary canal from a finished pharmaceutical product adapted for delivery of the aromatic-cationic peptide, wherein an outer surface of the product is substantially free of an acid resistant protective vehicle, wherein the pharmaceutical product is released into the alimentary canal in a quantity which, if added to 10 milliliters of 0.1M aqueous sodium bicarbonate solution, would be sufficient to lower pH of the solution to no higher than 5.5.

In some embodiments, the therapeutic aromatic-cationic peptide, the at least one pH-lowering agent and the at least one absorption enhancer are released from the finished pharmaceutical product more rapidly than from a corresponding pharmaceutical composition comprising an acid resistant protective vehicle. In some embodiments, a maximum plasma concentration of the aromatic-cationic peptide is achieved in the subject in 60 minutes or less. In some embodiments, the pH-lowering agent is present in a quantity which, if all ingredients were added to 10 milliliters of 0.1M aqueous sodium bicarbonate solution, would be sufficient to lower the pH of the solution to no higher than 3.5. In some embodiments, the absorption enhancer is selected from the group consisting of a cationic surfactant and an anionic surfactant that is a cholesterol derivative. In some embodiments, the pH-lowering agent has a pKa no higher than 4.2 and a solubility in water of at least 30 grams per 100 milliliters of water at room temperature. In some embodiments, the pH-lowering agent is present in an amount of not less than 300 milligrams.

In some embodiments, the aromatic-cationic peptide comprises the amino acid sequence Phe-D-Arg-Phe-Lys-NH₂ or a pharmaceutically acceptable salt thereof, such as acetate salt or trifluoroacetate salt. In some embodiments, the aromatic-cationic peptide comprises the amino acid sequence D-Arg-2′6′-Dmt-Lys-Phe-NH₂ or a pharmaceutically acceptable salt thereof, such as acetate salt or trifluoroacetate salt. In some embodiments, the aromatic-cationic peptide is selected from the group consisting of:

Phe-Arg-D-His-Asp
Met-Tyr-D-Lys-Phe-Arg
Phe-D-Arg-His
Tyr-D-Arg-Phe-Lys-NH₂
2′6′-Dmt-D-Arg-Phe-Lys-NH₂
2′6′-Dmt-D-Arg-Phe Orn-NH₂
2′6′-Dmt-D-Cit-Phe Lys-NH₂
Phe-D-Arg-2′6′-Dmt-Lys-NH₂
2′6′-Dmt-D-Arg-Phe-Ahp-NH₂
H-Phe-D-Arg-Phe-Lys-Cys-NH₂
2′6′-Dmp-D-Arg-2′6′-Dmt-Lys-NH₂
2′6′-Dmp-D-Arg-Phe-Lys-NH₂
Tyr-Arg-Phe-Lys-Glu-His-Trp-D-Arg
Lys-Gln-Tyr-D-Arg-Phe-Trp
D-Arg-2′6′-Dmt-Lys-Trp-NH₂
D-Arg-Trp-Lys-Trp-NH₂
D-Arg-2′6′-Dmt-Lys-Phe-Met-NH₂
D-Arg-2′6′-Dmt-Lys(N^{α}Me)-Phe-NH₂
D-Arg-2′6′-Dmt-Lys-Phe(NMe)—NH₂
D-Arg-2′6′-Dmt-Lys(N^{α}Me)-Phe(NMe)—NH₂
D-Arg(N^{α}Me)-2′6′-Dmt(NMe)-Lys(N^{α}Me)-Phe(NMe)—NH₂
D-Arg-2′6′-Dmt-Lys-Phe-Lys-Trp-NH₂
D-Arg-2′6′-Dmt-Lys-2′6′-Dmt-Lys-Trp-NH₂
D-Arg-2′6′-Dmt-Lys-Phe-Lys-Met-NH₂
D-Arg-2′6′-Dmt-Lys-2′6′-Dmt-Lys-Met-NH₂
D-Arg-2′6′-Dmt-Lys-Phe-Sar-Gly-Cys-NH₂
D-Arg-Ψ[CH₂—NH]2′6′-Dmt-Lys-Phe-NH₂
D-Arg-2′6′-Dmt-Ψ[CH₂—NH]Lys-Phe-NH₂
D-Arg-2′6′-Dmt-LysΨ[CH₂—NH]Phe-NH₂
D-Arg-2′6′-Dmt-Ψ[CH₂—NH]Lys-Ψ[CH₂—NH]Phe-NH₂
Lys-D-Arg-Tyr-NH₂
D-Tyr-Trp-Lys-NH₂
Trp-D-Lys-Tyr-Arg-NH₂
Tyr-His-D-Gly-Met
Tyr-D-Arg-Phe-Lys-Glu-NH₂
Met-Tyr-D-Arg-Phe-Arg-NH₂
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr-D-Phe-Glu-D-Asp-D-Asp-D-His-D-Lys-Arg-Trp-NH₂
Ala-D-Phe-D-Arg-Tyr-Lys-D-Trp-His-D-Tyr-Gly-Phe
Tyr-D-His-Phe-D-Arg-Asp-Lys-D-Arg-His-Trp-D-His-Phe
Phe-Phe-D-Tyr-Arg-Glu-Asp-D-Lys-Arg-D-Arg-His-Phe-NH₂
Phe-Tyr-Lys-D-Arg-Trp-His-D-Lys-D-Lys-Glu-Arg-D-Tyr-Thr
Tyr-Asp-D-Lys-Tyr-Phe-D-Lys-D-Arg-Phe-Pro-D-Tyr-His-Lys
Glu-Arg-D-Lys-Tyr-D-Val-Phe-D-His-Trp-Arg-D-Gly-Tyr-Arg-D-Met-NH₂
Arg-D-Leu-D-Tyr-Phe-Lys-Glu-D-Lys-Arg-D-Trp-Lys-D-Phe-Tyr-D-Arg-Gly
D-Glu-Asp-Lys-D-Arg-D-His-Phe-Phe-D-Val-Tyr-Arg-Tyr-D-Tyr-Arg-His-Phe-NH₂
Asp-Arg-D-Phe-Cys-Phe-D-Arg-D-Lys-Tyr-Arg-D-Tyr-Trp-D-His-Tyr-D-Phe-Lys-Phe
His-Tyr-D-Arg-Trp-Lys-Phe-D-Asp-Ala-Arg-Cys-D-Tyr-His-Phe-D-Lys-Tyr-His-Ser-NH₂
Gly-Ala-Lys-Phe-D-Lys-Glu-Arg-Tyr-His-D-Arg-D-Arg-Asp-Tyr-Trp-D-His-Trp-His-D-Lys-Asp
Thr-Tyr-Arg-D-Lys-Trp-Tyr-Glu-Asp-D-Lys-D-Arg-His-Phe-D-Tyr-Gly-Val-Ile-D-His-Arg-
Tyr-Lys-NH₂

The present disclosure provides a therapeutically effective oral pharmaceutical composition for reliably delivering pharmaceutical peptides, e.g., physiologically active peptides such as aromatic-cationic peptides of the present technology, as well as polypeptides such as insulin, salmon calcitonin, vasopressin, and others discussed herein. The disclosure further provides therapeutic methods for enhancing the bioavailability of such peptides.

This disclosure further provides methods of treating medical conditions and diseases by administering aromatic-cationic peptide of the present technology such as D-Arg-Dmt-Lys-Phe-NH₂ alone or in conjunction with one or more other peptide therapeutics.

In one aspect, the disclosure provides a pharmaceutical composition for oral delivery of aromatic-cationic peptides of the present technology comprising: (A) a therapeutically effective amount of the aromatic-cationic peptide linked to a membrane translocator, wherein the membrane translocator is capable of being at least partially cleaved by a blood or lymphatic system protease; (B) at least one pharmaceutically acceptable pH-lowering agent and/or protease inhibitor; and (C) an acid resistant protective vehicle effective to transport the pharmaceutical composition through the stomach of a patient while preventing contact between the aromatic-cationic peptide and stomach proteases.

Therapeutic peptides include but are not limited to aromatic-cationic peptides of the present technology, as well as polypeptides such as insulin, vasopressin salmon calcitonin, glucagon-like peptide 1, parathyroid hormone, luteinizing hormone releasing hormone, erythropoietin, and analogs thereof.

In another aspect, this disclosure provides a method for enhancing the bioavailability of a aromatic-cationic peptide delivered orally, the method comprising: (A) linking the aromatic-cationic peptide to a membrane translocator capable of being at least partially cleaved by a plasma protease; and (B) selectively releasing the peptide linked to the membrane translocator, together with at least one pH-lowering agent and/or protease inhibitor into a patient's intestine following passage of the peptide, pH-lowering agent and/or protease inhibitor through the patient's mouth and stomach under protection of an acid resistant protective vehicle which substantially prevents contact between stomach proteases and the peptide.

The present methods reduce the likelihood of proteolytic degradation of aromatic-cationic peptides of the present technology by simultaneously protecting the peptides from proteolytic attack by (1) stomach proteases which are typically most active at acidic pHs and (2) intestinal or pancreatic proteases (which are typically most active at basic to neutral pH). The methods promote the process by which the therapeutic peptides cross the intestinal brush border membrane into the blood due to the presence of the membrane translocator, while continuing to protect the peptide from proteolytic degradation.

An acid resistant protective vehicle protects the aromatic-cationic peptide from the acid-acting proteases of the stomach. Significant quantities of acid (with which the peptide active agent is intermixed) then reduce the activity of neutral to basic-acting proteases in the intestine (e.g., luminal or digestive protease and proteases of the brush border membrane) by lowering pH below the optimal activity range of these intestinal proteases.

The membrane translocator capable when linked to the aromatic-cationic peptide enhances transport of the peptide through intestinal mucous layers, through the brush border membrane and into the blood. Subsequently, the membrane translocator is cleaved by a blood or lymphatic system protease, thus releasing the aromatic-cationic peptide in a patient's system.

The present disclosure provides peptide pharmaceutical compositions which, when administered nasally, provide good bioavailability of aromatic-cationic peptides of the present technology, resulting in a significant increase in blood concentration of the peptide. The disclosure further provides aromatic-cationic peptide pharmaceutical compositions that are well-tolerated when administered to the nasal mucosa.

In one embodiment, the disclosure provides a pharmaceutical composition for nasal delivery of an aromatic-cationic peptide comprising: (1) the aromatic-cationic peptide; and (2) a bioavailability enhancing agent selected from the group consisting of a fatty acid, a sugar ester of a fatty acid and a mixture thereof.

In another embodiment, the disclosure provides a pharmaceutical composition for nasal delivery of an aromatic-cationic peptide comprising: (1) the aromatic-cationic peptide; (2) a sugar ester of a fatty acid; and (3) an acyl carnitine.

In another embodiment, the disclosure provides a pharmaceutical composition for nasal delivery of an aromatic-cationic peptide comprising: (1) the aromatic-cationic peptide of the present technology; (2) oleic acid; (3) sucrose laurate; (4) a citrate-based bioavailability enhancing agent selected from the group consisting of citric acid, citric acid salt and a mixture of citric acid and citric acid salt; wherein the pharmaceutical composition is an aqueous solution buffered to a select pH range. In one embodiment, the disclosure provides a pharmaceutical composition for nasal delivery as described above, wherein the pH range is no lower than 3.0 and no higher than 6.5. In one embodiment, the disclosure provides a pharmaceutical composition for nasal delivery as described above, wherein the pH range is no lower than 2.0 and no higher than 7.5. In another embodiment, the disclosure provides a pharmaceutical composition for nasal delivery as described above, wherein the pH range is no lower than 1.5 and no higher than 10.0.

In another embodiment, the disclosure provides a pharmaceutical composition for nasal delivery of an aromatic-cationic peptide comprising: (1) the aromatic-cationic peptide; (2) L-lauroyl carnitine; (3) sucrose laurate; (4) a citrate-based bioavailability enhancing agent selected from the group consisting of citric acid, citric acid salt and a mixture of citric acid and citric acid salt; wherein the pharmaceutical composition is an aqueous solution buffered to a select pH range. In one embodiment, the disclosure provides a pharmaceutical composition for nasal delivery as described above, wherein the pH range is no lower than 3.0 and no higher than 6.5. In one embodiment, the disclosure provides a pharmaceutical composition for nasal delivery as described above, wherein the pH range is no lower than 2.0 and no higher than 7.5. In another embodiment, the disclosure provides a pharmaceutical composition for nasal delivery as described above, wherein the pH range is no lower than 1.5 and no higher than 10.0.

In some embodiments, the present disclosure provides a liquid pharmaceutical composition comprising an aromatic-cationic peptide or an acid addition salt thereof and citric acid and/or salt thereof in a concentration from about 10 to about 50 mM, the composition being in a form suitable for nasal administration.

The present disclosure also provides a liquid pharmaceutical composition comprising aromatic-cationic peptide, about 10 mM citric acid, about 0.2% phenylethyl alcohol, about 0.5% benzyl alcohol, and about 0.1% Tween 80.

The present disclosure further provides a liquid pharmaceutical composition comprising aromatic-cationic peptide of the present technology such as D-Arg-Dmt-Lys-Phe-NH₂, about 20 mM citric acid, about 0.2% phenylethyl alcohol, about 0.5% benzyl alcohol, and about 0.1% Tween 80.

The present disclosure also provides a method of administering an aromatic-cationic peptide to a subject requiring aromatic-cationic peptide treatment, which method comprises administering via the nasal route to the subject a liquid pharmaceutical composition comprising aromatic-cationic peptide or an acid addition salt thereof and citric acid or salt thereof in a concentration from about 10 to about 50 mM.

The present disclosure further provides a method of improving the stability of a liquid pharmaceutical composition of aromatic-cationic peptide comprising adding citric acid or a salt thereof in a concentration from about 10 to about 50 mM to the composition.

The present disclosure also provides a method of improving the bioavailability or the concentration of plasma aromatic-cationic peptide in a subject following nasal administration of a liquid pharmaceutical composition of aromatic-cationic peptide, which method comprises adding citric acid or a salt thereof in a concentration from about 10 to about 50 mM to the composition prior to the administration.

In some embodiments, the present disclosure relates to the administration of aromatic-cationic peptides of the present technology in conjunction with peptides for appetite suppression and weight control. In some embodiments, the peptide for appetite suppression and weight control is an calcitonin analog. In some embodiments, the peptide has the amino acid sequence Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Arg-Thr-Xaa-Xaa-Gly-Xaa-Xaa-Thr-Xaa, wherein amino acids 26, 27, 28, 29, and 31 can be any naturally occurring amino acid, and wherein amino acid 31 is optionally amidated.

### DETAILED DESCRIPTION

### I. Aromatic-Cationic Peptides

Aromatic-cationic peptides which may benefit from oral delivery in accordance with the present technology include aromatic-cationic peptides that are physiologically active and have a plurality of amino acids and at least one peptide bond in its molecular structure. The present formulations, by several mechanisms, suppress the degradation of the active ingredients (e.g., aromatic-cationic peptides) by protease that would otherwise tend to cleave one or more of the peptide bonds of the active ingredient. The molecular structure may further include other constituents or modifications. Both man-made and natural peptides can be orally delivered in accordance with the present technology.

In some aspects, the present technology provides an aromatic-cationic peptide or a pharmaceutically acceptable salt thereof such as acetate salt or trifluoroacetate salt. In some embodiments, the peptide comprises

at least one net positive charge;
a minimum of three amino acids;
a maximum of about twenty amino acids;
a relationship between the minimum number of net positive charges (pₘ) and the total number of amino acid residues (r) wherein 3pₘ is the largest number that is less than or equal to r+1; and
a relationship between the minimum number of aromatic groups (a) and the total number of net positive charges (pₜ) wherein 2a is the largest number that is less than or equal to pₜ+1, except that when a is 1, pₜ may also be 1.

In some embodiments, the peptide comprises the amino acid sequence Phe-D-Arg-Phe-Lys-NH₂ or D-Arg-2′6′-Dmt-Lys-Phe-NH₂. In some embodiments, the peptide comprises one or more of:

Phe-Arg-D-His-Asp
Met-Tyr-D-Lys-Phe-Arg
Phe-D-Arg-His
Tyr-D-Arg-Phe-Lys-NH₂
2′6′-Dmt-D-Arg-Phe-Lys-NH₂
2′6′-Dmt-D-Arg-Phe Orn-NH₂
2′6′-Dmt-D-Cit-Phe Lys-NH₂
Phe-D-Arg-2′6′-Dmt-Lys-NH₂
2′6′-Dmt-D-Arg-Phe-Ahp-NH₂
H-Phe-D-Arg-Phe-Lys-Cys-NH₂
2′6′-Dmp-D-Arg-2′6′-Dmt-Lys-NH₂
2′6′-Dmp-D-Arg-Phe-Lys-NH₂
Tyr-Arg-Phe-Lys-Glu-His-Trp-D-Arg
Lys-Gln-Tyr-D-Arg-Phe-Trp
D-Arg-2′6′-Dmt-Lys-Trp-NH₂
D-Arg-Trp-Lys-Trp-NH₂
D-Arg-2′6′-Dmt-Lys-Phe-Met-NH₂
D-Arg-2′6′-Dmt-Lys(N^{α}Me)-Phe-NH₂
D-Arg-2′6′-Dmt-Lys-Phe(NMe)—NH₂
D-Arg-2′6′-Dmt-Lys(N^{α}Me)-Phe(NMe)—NH₂
D-Arg(N^{α}Me)-2′6′-Dmt(NMe)-Lys(N^{α}Me)-Phe(NMe)—NH₂
D-Arg-2′6′-Dmt-Lys-Phe-Lys-Trp-NH₂
D-Arg-2′6′-Dmt-Lys-2′6′-Dmt-Lys-Trp-NH₂
D-Arg-2′6′-Dmt-Lys-Phe-Lys-Met-NH₂
D-Arg-2′6′-Dmt-Lys-2′6′-Dmt-Lys-Met-NH₂
D-Arg-2′6′-Dmt-Lys-Phe-Sar-Gly-Cys-NH₂
D-Arg-Ψ[CH₂—NH]2′6′-Dmt-Lys-Phe-NH₂
D-Arg-2′6′-Dmt-Ψ[CH₂—NH]Lys-Phe-NH₂
D-Arg-2′6′-Dmt-LysΨ[CH₂—NH]Phe-NH₂
D-Arg-2′6′-Dmt-Ψ[CH₂—NH]Lys-Ψ[CH₂—NH]Phe-NH₂
Lys-D-Arg-Tyr-NH₂
D-Tyr-Trp-Lys-NH₂
Trp-D-Lys-Tyr-Arg-NH₂
Tyr-His-D-Gly-Met
Tyr-D-Arg-Phe-Lys-Glu-NH₂
Met-Tyr-D-Arg-Phe-Arg-NH₂
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr-D-Phe-Glu-D-Asp-D-Asp-D-His-D-Lys-Arg-Trp-NH₂
Ala-D-Phe-D-Arg-Tyr-Lys-D-Trp-His-D-Tyr-Gly-Phe
Tyr-D-His-Phe-D-Arg-Asp-Lys-D-Arg-His-Trp-D-His-Phe
Phe-Phe-D-Tyr-Arg-Glu-Asp-D-Lys-Arg-D-Arg-His-Phe-NH₂
Phe-Tyr-Lys-D-Arg-Trp-His-D-Lys-D-Lys-Glu-Arg-D-Tyr-Thr
Tyr-Asp-D-Lys-Tyr-Phe-D-Lys-D-Arg-Phe-Pro-D-Tyr-His-Lys
Glu-Arg-D-Lys-Tyr-D-Val-Phe-D-His-Trp-Arg-D-Gly-Tyr-Arg-D-Met-NH₂
Arg-D-Leu-D-Tyr-Phe-Lys-Glu-D-Lys-Arg-D-Trp-Lys-D-Phe-Tyr-D-Arg-Gly
D-Glu-Asp-Lys-D-Arg-D-His-Phe-Phe-D-Val-Tyr-Arg-Tyr-D-Tyr-Arg-His-Phe-NH₂
Asp-Arg-D-Phe-Cys-Phe-D-Arg-D-Lys-Tyr-Arg-D-Tyr-Trp-D-His-Tyr-D-Phe-Lys-Phe
His-Tyr-D-Arg-Trp-Lys-Phe-D-Asp-Ala-Arg-Cys-D-Tyr-His-Phe-D-Lys-Tyr-His-Ser-NH₂
Gly-Ala-Lys-Phe-D-Lys-Glu-Arg-Tyr-His-D-Arg-D-Arg-Asp-Tyr-Trp-D-His-Trp-His-D-Lys-Asp
Thr-Tyr-Arg-D-Lys-Trp-Tyr-Glu-Asp-D-Lys-D-Arg-His-Phe-D-Tyr-Gly-Val-Ile-D-His-Arg-
Tyr-Lys-NH₂

In one embodiment, the aromatic-cationic peptide is defined by formula I.

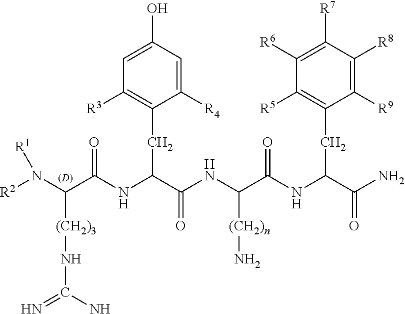

wherein R¹ and R² are each independently selected from

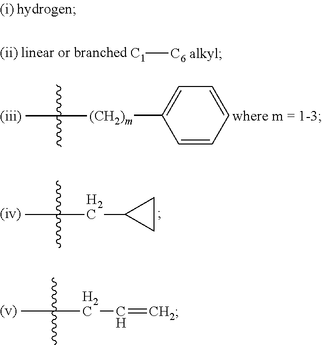

R³ and R⁴ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo;

R⁵, R⁶, R⁷, R⁸, and R⁹ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and

n is an integer from 1 to 5.

In a particular embodiment, R¹ and R² are hydrogen; R³ and R⁴ are methyl; R⁵, R⁶, R⁷, R⁸, and R⁹ are all hydrogen; and n is 4.

In one embodiment, the peptide is defined by formula II:

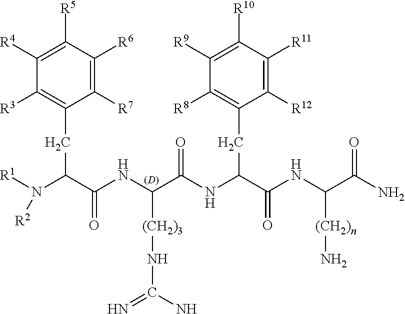

wherein R¹ and R² are each independently selected from

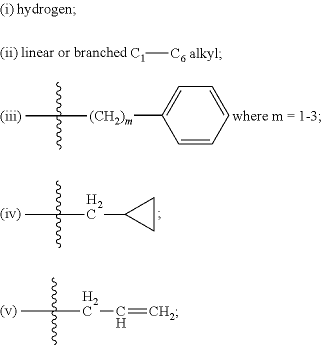

R³, R⁴, R⁵, R⁶, R⁷, R⁸, R⁹, R¹⁰, R¹¹ and R¹² are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and

n is an integer from 1 to 5.

In a particular embodiment, R¹, R², R³, R⁴, R⁵, R⁶, R⁷, R⁸, R⁹, R¹⁰, R¹¹, and R¹² are all hydrogen; and n is 4. In another embodiment, R¹, R², R³, R⁴, R⁵, R⁶, R⁷, R⁸, R⁹, and R¹¹ are all hydrogen; R⁸ and R¹² are methyl; R¹⁰ is hydroxyl; and n is 4.

In one embodiment, the aromatic-cationic peptides of the present technology have a core structural motif of alternating aromatic and cationic amino acids. Fr example, the peptide may be a tetrapeptide defined by any of formulas III to VI set forth below:

Aromatic-Cationic-Aromatic-Cationic (Formula III)

Cationic-Aromatic-Cationic-Aromatic (Formula IV)

Aromatic-Aromatic-Cationic-Cationic (Formula V)

Cationic-Cationic-Aromatic-Aromatic (Formula VI)

wherein, Aromatic is a residue selected from the group consisting of: Phe (F), Tyr (Y), Trp (W), and Cyclohexylalanine (Cha); and Cationic is a residue selected from the group consisting of: Arg (R), Lys (K), Norleucine (Nle), and 2-amino-heptanoic acid (Ahe).

The peptides disclosed herein may be formulated as pharmaceutically acceptable salts. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucoronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the salt is an acetate salt. Additionally or alternatively, in other embodiments, the salt is a trifluoroacetate salt.

The aromatic-cationic peptides of the present technology disclosed herein may be synthesized by any of the methods well known in the art. Suitable methods for chemically synthesizing the protein include, for example, liquid phase and solid phase synthesis, and those methods described by Stuart and Young in Solid Phase Peptide Synthesis, Second Edition, Pierce Chemical Company (1984), and in Methods Enzymol., 289, Academic Press, Inc, New York (1997). Recombinant peptides may be generated using conventional techniques in molecular biology, protein biochemistry, cell biology, and microbiology, such as those described in Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

Additional peptide active compounds of the present technology include, but are not limited to, polypeptides such as insulin, vasopressin and calcitonin. Other examples include, but are not limited to, calcitonin gene-related peptide, parathyroid hormone (full length or truncated, amidated or in the free acid form, further modified or not), luteinizing hormone-releasing factor, erythropoietin, tissue plasminogen activators, human growth hormone, adrenocorticototropin, various interleukins, enkephalin, DALDA derivatives such as dmt-DALDA and the like. Many others are known in the art. It is expected that any pharmaceutical compound having peptide bonds which would be subject to cleavage in the gastrointestinal tract would benefit from oral delivery in accordance with the present technology because of the reduction in such cleavage that is afforded by the present formulations.

In some embodiments, the aromatic-cationic peptide comprises the sequence Phe-D-Arg-Phe-Lys-NH₂ and/or D-Arg-2′6′-Dmt-Lys-Phe-NH₂. In some embodiments, the aromatic-cationic peptide comprises from 0.02 to 0.2 percent by weight relative to the total weight of the overall pharmaceutical composition. Other aromatic-cationic peptides of the present technology may be present at higher or lower concentrations depending on desired target blood concentrations for the peptide and its bioavailability in the oral delivery system of the present technology

Aromatic-cationic peptide precursors may be made by either chemical (e.g., using solution and solid phase chemical peptide synthesis) or recombinant syntheses known in the art. Precursors of other amidated aromatic-cationic peptides of the present technology may be made in like manner. Recombinant production is believed significantly more cost effective. Precursors are converted to active peptides by amidation reactions that are also known in the art. For example, enzymatic amidation is described in U.S. Pat. No. 4,708,934 and European Patent Publications 0 308 067 and 0 382 403. Recombinant production can be used for both the precursor and the enzyme that catalyzes the conversion of the precursor to the desired active form of the aromatic-cationic peptide. Such recombinant production is discussed in Biotechnology, Vol. 11 (1993) pp. 64-70, which further describes a conversion of a precursor to an amidated product. During amidation, a keto-acid such as an alpha-keto acid, or salt or ester thereof, wherein the alpha-keto acid has the molecular structure RC(O)C(O)OH, and wherein R is selected from the group consisting of aryl, a C1-C4 hydrocarbon moiety, a halogenated or hydroxylated C1-C4 hydrocarbon moiety, and a C1-C4 carboxylic acid, may be used in place of a catalase co-factor. Examples of these keto acids include, but are not limited to, ethyl pyruvate, pyruvic acid and salts thereof, methyl pyruvate, benzoyl formic acid and salts thereof, 2-ketobutyric acid and salts thereof, 3-methyl-2-oxobutanoic acid and salts thereof, and 2-keto glutaric acid and salts thereof.

The production of the recombinant aromatic-cationic peptide may proceed, for example, by producing glycine-extended precursor in E. coli as a soluble fusion protein with glutathione-S-transferase. An α-amidating enzyme catalyzes conversion of precursors to active aromatic-cationic peptide. That enzyme is recombinantly produced, for example, in Chinese Hamster Ovary (CHO) cells as described in the Biotechnology article cited above. Other precursors to other amidated peptides may be produced in like manner. Peptides that do not require amidation or other additional functionalities may also be produced in like manner. Other peptide active agents are commercially available or may be produced by techniques known in the art.

### II. Oral Delivery of Peptide Pharmaceutical Compositions

It has surprisingly been found that administering the pharmaceutical formulations of this technology, without an enteric coating, increases the speed of peptide absorption (relative to corresponding enteric-coated pharmaceuticals) without reducing bioavailability below practical levels. While some reduction in bioavailability does occur, this reduction is not expected to preclude effective medical treatment, or to unduly detract from the advantages of greater speed, especially in applications where such speed is particularly advantageous, i.e., in the case of pain relief. The present formulations permit more rapid absorption of the active aromatic-cationic peptides of the present technology or pharmaceutically acceptable salts thereof, such as acetate salt or trifluoroacetate salt, due to the reduction in the time necessary for the vehicle (e.g., a capsule or tablet) to be dissolved and the active ingredients to be released. It also permits such release further upstream in the alimentary canal, e.g., in the esophagus and/or stomach, instead of awaiting passage of the material into the intestine. See e.g., U.S. Patent Publication No. 2005/0282756 and U.S. Patent Publication No. 2007/0134279, herein incorporated by reference in their entirety.

In accordance with the present technology, subjects in need of treatment with aromatic-cationic peptide active ingredients are provided with a finished pharmaceutical product, optionally in tablet form of an ordinary size in the pharmaceutical industry, formed of an oral pharmaceutical composition comprising one or more of such peptide active ingredients (at appropriate dosage). The finished pharmaceutical product may additionally be prepared, if desired, in (for example) capsule form. The dosages and frequency of administering the products are discussed in more detail below. Subjects who may benefit are any who suffer from disorders that respond favorably to increased levels of a peptide-containing compound.

The oral peptide formulations described herein are useful in the treatment of disorders stemming from or related to mitochondrial permeability transition (MPT) and/or cellular oxidative damage. For example, oral peptide formulations of the aromatic-cationic peptides of the present technology Phe-D-Arg-Phe-Lys-NH₂ and D-Arg-2′6′-Dmt-Lys-Phe-NH₂, or pharmaceutically acceptable salts thereof, may be used to treat subjects suffering from vascular occlusion, kidney ischemia, tissue ischemia-reperfusion injury, acute myocardial infarction, diseases or disorders of the eye, or neurological disorders such as Alzheimer's and Parkinson's diseases. Pharmaceutically acceptable salts include, but are not limited to, e.g., acetate salt and trifluoroacetate salt.

Not wishing to be bound by theory, the pharmaceutical formulations described herein are believed to overcome a series of different and unrelated natural barriers to bioavailability. Various components of the pharmaceutical compositions act to overcome different barriers by mechanisms appropriate to each, and result in synergistic effects on the bioavailability of a peptide active ingredient. As discussed below, inherent physical and chemical properties of peptides make certain absorption enhancers more effective than others in boosting its bioavailability.

The aromatic-cationic peptide active compound of the present technology is contained within a formulation adopted for oral administration. In accordance with the present technology, proteolytic degradation of the peptide by stomach proteases (most of which are active in the acid pH range) is reduced due to administration of the formulation to the patient on an empty stomach (although this is not required in order to achieve adequate results), while degradation by intestinal or pancreatic proteases (most of which are active in the neutral to basic pH range) is reduced due to the effect of the pH lowering agent in adjusting the pH of the intestinal environment to sub-optimal levels. Solubility enhancers aid passage of the aromatic-cationic peptide through the intestinal epithelial barrier.

The pH-lowering agent is believed to lower the local pH (where the active agent has been released) to levels below the optimal range for many intestinal proteases. This decrease in pH reduces the proteolytic activity of the intestinal proteases, thus affording protection to the peptide from potential degradation should the peptide be present within the intestine. The activity of these proteases is diminished by the temporarily acidic environment as discussed herein. For example, sufficient acid should be provided that local intestinal pH is lowered temporarily to 5.5 or below, 4.7 or below, or 3.5 or below. The sodium bicarbonate test described below (in the section captioned “the pH-Lowering Agent”) is indicative of the required acid amount. Conditions of reduced pH should persist for a time period sufficient to protect the aromatic-cationic peptide from proteolytic degradation until at least some of the aromatic-cationic peptide has had an opportunity to cross into the bloodstream. By way of example, but not by way of limitation, for salmon calcitonin, a 32 amino acid peptide, experiments have demonstrated Tₘₐₓ of 5-15 minutes for blood levels of salmon calcitonin when the active components are injected directly into the duodenum, ilium or colon. The absorption enhancers of the present formulations synergistically promote peptide absorption into the blood while conditions of reduced proteolytic activity prevail. The mechanism by which the present formulations are believed to accomplish the goal of enhanced bioavailability is aided by having active components of the finished pharmaceutical product released together as simultaneously as possible.

The absorption enhancer, which may be a solubility enhancer and/or transport enhancer (as described in more detail below), aids transport of the aromatic-cationic peptide from the alimentary canal into the blood, and may promote the process so that it better occurs during the time period of reduced intestinal pH and reduced intestinal proteolytic activity. Many surface active agents may act as both solubility enhancers and transport (uptake) enhancers. Again without intending to be bound by theory, it is believed that enhancing solubility provides (1) a more simultaneous release of the active components of the present formulations into the aqueous portion of the alimentary tract, (2) better solubility of the peptide in, and transport through, a mucous layer such as that found along the intestinal walls. Once the peptide active ingredient reaches, e.g., the intestinal walls, an uptake enhancer provides better transport through the brush border membrane of the intestine into the blood, via either transcellular or paracellular transport. As discussed in more detail below, many compounds may provide both functions. In those instances, embodiments utilizing both of these functions may do so by adding only one additional compound to the pharmaceutical composition. In other embodiments, separate absorption enhancers may provide the two functions separately.

Each of the ingredients of the finished pharmaceutical product of the present technology is separately discussed below. Combinations of multiple pH-lowering agents, or multiple enhancers can be used as well as using just a single pH-lowering agent and/or single enhancer. Some combinations are also discussed below.

Without intending to be bound by theory, the pharmaceutical formulations of the present technology are believed to overcome a series of different and unrelated natural barriers to bioavailability. Various components of the pharmaceutical compositions act to overcome different barriers by mechanisms appropriate to each, and result in synergistic effects on the bioavailability of a peptide active ingredient. As discussed below, inherent physical and chemical properties of peptides make certain absorption enhancers more effective than others in boosting its bioavailability.

The aromatic-cationic peptide active compound (or a pharmaceutically acceptable salt thereof, such as acetate salt or trifluoroacetate salt) is contained within a formulation adopted for oral administration. In accordance with the present technology, proteolytic degradation of the peptide by stomach proteases (most of which are active in the acid pH range) is reduced due to administration of the formulation to the patient on an empty stomach (although this is not required in order to achieve adequate results), while degradation by intestinal or pancreatic proteases (most of which are active in the neutral to basic pH range) is reduced due to the effect of the pH lowering agent in adjusting the pH of the intestinal environment to sub-optimal levels. Solubility enhancers aid passage of the aromatic-cationic peptide through the intestinal epithelial barrier.

### A. The pH-Lowering Agent

The total amount of the pH-lowering compound to be administered with each administration of aromatic-cationic peptide should be an amount which, when released into the intestine for example, is sufficient to lower the local intestinal pH substantially below the pH optima for proteases found there. The quantity required will necessarily vary with several factors including the type of pH-lowering agent used (discussed below) and the equivalents of protons provided by a given pH-lowering agent. In practice, the amount required to provide good bioavailability is an amount which, when the pharmaceutical product of the present technology is added to a solution of 10 milliliters of 0.1 M sodium bicarbonate, lowers the pH of that sodium bicarbonate solution to no higher than 5.5, no higher than 4.7, or no higher than 3.5. Enough acid to lower pH, in the foregoing test, to about 2.8 has been used in some embodiments. At least 300 milligrams or at least 400 milligrams of the pH-lowering agent are used in the pharmaceutical composition of the present technology. The foregoing values relate to the total combined weight of all pH-lowering agents where two or more of such agents are used in combination. The oral formulation should not include an amount of any base which, when released together with the pH-lowering compound, would prevent the pH of the above-described sodium bicarbonate test from dropping to 5.5 or below.

The pH-lowering agent of the present formulations may be any pharmaceutically acceptable compound that is not toxic in the gastrointestinal tract and is capable of either delivering hydrogen ions (a traditional acid) or of inducing higher hydrogen ion content from the local environment. It may also be any combination of such compounds. In some embodiments, at least one pH-lowering agent used in the present formulations has a pKa no higher than 4.2, or no higher than 3.0. In some embodiments, the pH lowering agent has a solubility in water of at least 30 grams per 100 milliliters of water at room temperature.

Examples of compounds that induce higher hydrogen ion content include aluminum chloride and zinc chloride. Pharmaceutically acceptable traditional acids include, but are not limited to acid salts of amino acids (e.g. amino acid hydrochlorides) or derivatives thereof Examples of these are acid salts of acetylglutamic acid, alanine, arginine, asparagine, aspartic acid, betaine, carnitine, carnosine, citrulline, creatine, glutamic acid, glycine, histidine, hydroxylysine, hydroxyproline, hypotaurine, isoleucine, leucine, lysine, methylhistidine, norleucine, ornithine, phenylalanine, proline, sarcosine, serine, taurine, threonine, tryptophan, tyrosine and valine.

Other examples of useful pH-lowering compounds include dicarboxylic and tricarboxylic carboxylic acids. Acids such as acetylsalicylic, acetic, ascorbic, citric, fumaric, glucuronic, glutaric, glyceric, glycocolic, glyoxylic, isocitric, isovaleric, lactic, maleic, oxaloacetic, oxalosuccinic, propionic, pyruvic, succinic, tartaric, valeric, and the like have been found useful.

Other useful pH-lowering agents that might not usually be called “acids” in the art, but which may nonetheless be useful in accordance with the present technology are phosphate esters (e.g., fructose 1,6diphosphate, glucose 1,6diphosphate, phosphoglyceric acid, and diphosphoglyceric acid). CARBOPOL™ (Trademark of BF Goodrich) and polymers such as polycarbophil may also be used to lower pH.

Any combination of pH lowering agent that achieves the required pH level of no higher than 5.5 in the sodium bicarbonate test discussed above may be used. One embodiment utilizes, as at least one of the pH-lowering agents in the finished pharmaceutical product, an acid selected from the group consisting of citric acid, tartaric acid and an acid salt of an amino acid.

When aromatic-cationic peptides of the present technology or a pharmaceutically acceptable salt thereof, such as acetate salt or trifluoroacetate salt, are the active agent, certain ratios of pH-lowering agent to peptide may prove especially effective. For example, in some embodiments, the weight ratio of pH-lowering agent to aromatic-cationic peptide exceed 200:1, 800:1, or 2000:1. In some embodiments, the weight ratio of pH-lowering agent to aromatic-cationic peptide exceeds 40:1, 400:1, or 4000:1.

### B. The Absorption Enhancer

The absorption enhancers are present in a quantity that constitutes from 0.1 to 20.0 percent by weight, relative to the overall weight of the pharmaceutical composition. Optimal absorption enhancers are surface active agents which act both as solubility enhancers and uptake enhancers. Generically speaking, “solubility enhancers” improve the ability of the components of the present formulations to be solubilized in either the aqueous environment into which they are originally released or into, for example, the lipophilic environment of the mucous layer lining the intestinal walls, or both. “Transport (uptake) enhancers” (which are frequently the same surface active agents used as solubility enhancers) are those which facilitate the ease by which aromatic-cationic peptides of the present technology cross the intestinal wall.

One or more absorption enhancers may perform one function only (e.g., solubility), or one or more absorption enhancers may perform the other function only (e.g., uptake), within the scope of the present technology. It is also possible to have a mixture of several compounds some of which provide improved solubility, some of which provide improved uptake and/or some of which perform both. Without intending to be bound by theory, it is believed that uptake enhancers may act by (1) increasing disorder of the hydrophobic region of the membrane exterior of cells, allowing for increased transcellular transport; or (2) leaching membrane proteins resulting in increased transcellular transport; or (3) widening pore radius between cells for increased paracellular transport.

Surface active agents are believed to be useful both as solubility enhancers and as uptake enhancers. For example, detergents are useful in (1) solubilizing all of the active components quickly into the aqueous environment where they are originally released, (2) enhancing lipophilicity of the components of the present formulations, especially the aromatic-cationic peptide, aiding its passage into and through the intestinal mucus, (3) enhancing the ability of the normally polar aromatic-cationic peptide to cross the epithelial barrier of the brush border membrane; and (4) increasing transcellular or paracellular transport as described above.

In some embodiments, when surface active agents are used as the absorption enhancers, the surface active agents are free flowing powders for facilitating the mixing and loading of capsules during the manufacturing process. Because of inherent characteristics of aromatic-cationic peptides of the present technology and other peptides (e.g., their isoelectric point, molecular weight, amino acid composition, etc.) certain surface active agents interact best with certain peptides. Indeed, some can undesirably interact with the charged portions of aromatic-cationic peptides of the present technology and prevent its absorption, thus undesirably resulting in decreased bioavailability. In some embodiments, when trying to increase the bioavailability of aromatic-cationic peptides of the present technology or other peptides, a surface active agent used as an absorption enhancer is selected from the group consisting of (i) anionic surface active agents that are cholesterol derivatives (e.g., bile acids), (ii) cationic surface agents (e.g., acyl carnitines, phospholipids and the like), (iii) non-ionic surface active agents, and (iv) mixtures of anionic surface active agents (especially those having linear hydrocarbon regions) together with negative charge neutralizers. Negative charge neutralizers include but are not limited to acyl carnitines, cetyl pyridinium chloride, and the like. In some embodiments, the absorption enhancer is soluble at acid pH, particularly in the 3.0 to 5.0 range.

In some embodiments, one combination useful with aromatic-cationic peptides of the present technology (or a pharmaceutically acceptable salt thereof, such as acetate salt or trifluoroacetate salt) mixes cationic surface active agents with anionic surface active agents that are cholesterol derivatives, and which are soluble at acid pH.

In some embodiments, a combination is an acid soluble bile acid together with a cationic surface active agent. In some embodiments, an acyl carnitine and sucrose ester is a good combination. In some embodiments, when a particular absorption enhancer is used alone, it comprises a cationic surface active agent. Acyl carnitines (e.g., lauroyl carnitine), phospholipids and bile acids are particularly good absorption enhancers, especially acyl carnitine. Anionic surfactants that are cholesterol derivatives are also used in some embodiments. It is the intent to avoid interactions with the aromatic-cationic peptide that interfere with absorption of aromatic-cationic peptide into the blood.

To reduce the likelihood of side effects, detergents, when used as the absorption enhancers of the present formulations, are either biodegradable or reabsorbable (e.g. biologically recyclable compounds such as bile acids, phospholipids, and/or acyl carnitines). Acylcarnitines are believed particularly useful in enhancing paracellular transport. When a bile acid (or another anionic detergent lacking linear hydrocarbons) is used in combination with a cationic detergent, aromatic-cationic peptides of the present technology are better transported both to and through the intestinal wall.

Absorption enhancers include: (a) salicylates such as sodium salicylate, 3-methoxysalicylate, 5-methoxysalicylate and homovanilate; (b) bile acids such as taurocholic, tauorodeoxycholic, deoxycholic, cholic, glycholic, lithocholate, chenodeoxycholic, ursodeoxycholic, ursocholic, dehydrocholic, fusidic, etc.; (c) non-ionic surfactants such as polyoxyethylene ethers (e.g. Brij 36T, Brij 52, Brij 56, Brij 76, Brij 96, Texaphor A6, Texaphor A14, Texaphor A60 etc.), p-t-octyl phenol polyoxyethylenes (Triton X-45, Triton X-100, Triton X-114, Triton X-305 etc.) nonylphenoxypoloxyethylenes (e.g. Igepal CO series), polyoxyethylene sorbitan esters (e.g. Tween-20, Tween-80 etc.); (d) anionic surfactants such as dioctyl sodium sulfosuccinate; (e) lyso-phospholipids such as lysolecithin and lysophosphatidylethanolamine; (f) acylcarnitines, acylcholines and acyl amino acids such as lauroylcarnitine, myristoylcarnitine, palmitoylcarnitine, lauroylcholine, myristoylcholine, palmitoylcholine, hexadecyllysine, N-acylphenylalanine, N-acylglycine etc.; g) water soluble phospholipids such as diheptanoylphosphatidylcholine, dioctylphosphatidylcholine etc.; (h) medium-chain glycerides which are mixtures of mono-, di- and triglycerides containing medium-chain-length fatty acids (caprylic, capric and lauric acids); (i) ethylene-diaminetetraacetic acid; (j) cationic surfactants such as cetylpyridinium chloride; (k) fatty acid derivatives of polyethylene glycol such as Labrasol, Labrafac, etc.; and (l) alkylsaccharides such as lauryl maltoside, lauroyl sucrose, myristoyl sucrose, palmitoyl sucrose, etc.

In some embodiments, and without intending to be bound by theory, cationic ion exchange agents (e.g. detergents) are included to provide solubility enhancement by another possible mechanism. In particular, they may prevent the binding of aromatic-cationic peptides of the present technology or other therapeutic agents to mucus. Cationic ion exchange agents include protamine chloride or any other polycation.

### C. Other Optional Ingredients

In some embodiments, a water-soluble barrier separate the pH-lowering agent from an acid resistant enteric coating. A conventional pharmaceutical capsule may, for example, be used for the purpose of providing this barrier. Many water soluble barriers are known in the art and include, but are not limited to, hydroxypropyl methylcellulose and conventional pharmaceutical gelatins.

In some embodiments, another peptide (such as albumin, casein, soy protein, other animal or vegetable proteins and the like) is included to reduce non-specific adsorption (e.g., binding of peptide to the intestinal mucus barrier) thereby lowering the necessary concentration of the aromatic-cationic peptide. When added, the peptide is from 1.0 to 10.0 percent by weight relative to the weight of the overall pharmaceutical composition. This second peptide should not be physiologically active and should be a food peptide such as soy bean peptide or the like. Without intending to be bound by theory, this second peptide may also increase bioavailability by acting as a protease scavenger that desirably competes with the aromatic-cationic peptide for protease interaction. The second peptide may also aid the active compound's passage through the liver.

All pharmaceutical compositions of the present technology may optionally also include common pharmaceutical diluents, glycants, lubricants, gelatin capsules, preservatives, colorants and the like in their usual known sizes and amounts.

### D. The Enteric Coating or Protective Vehicle

In some embodiments, aromatic-cationic peptide formulations include a carrier or vehicle that protects the formulation from stomach proteases. Any carrier or vehicle that protects the aromatic-cationic peptide from stomach proteases and then dissolves so that the other ingredients of the composition may be released in the intestine is suitable.

Many such enteric coatings are known in the art, and are useful in accordance with the present technology. Examples include cellulose acetate phthalate, hydroxypropyl methylethylcellulose succinate, hydroxypropyl methylcellulose phthalate, carboxylmethylethylcellulose and methacrylic acid-methyl methacrylate copolymer. In some embodiments, aromatic-cationic peptides of the present technology, absorption enhancers such as solubility and/or uptake enhancer(s), and pH-lowering compound(s), are included in a sufficiently viscous protective syrup to permit protected passage of the components of the composition through the stomach.

Suitable enteric coatings for protecting the aromatic-cationic peptide from stomach proteases may be applied, for example, to capsules after the remaining components have been loaded within the capsule. In other embodiments, enteric coating is coated on the outside of a tablet or coated on the outer surface of particles of active components which are then pressed into tablet form, or loaded into a capsule, which is itself coated with an enteric coating.

It is very desirable that all components of the present technology be released from the carrier or vehicle, and solubilized in the intestinal environment as simultaneously as possible. In some embodiments, the vehicle or carrier releases the active components in the small intestine where uptake enhancers that increase transcellular or paracellular transport are less likely to cause undesirable side effects than if the same uptake enhancers were later released in the colon. It is emphasized, however, that the present technology is believed effective in the colon as well as in the small intestine. Numerous vehicles or carriers, in addition to the ones discussed above, are known in the art. It is desirable keep the amount of enteric coating low. In some embodiments, the enteric coating adds no more than 30% to the weight of the remainder of pharmaceutical composition (the “remainder” being the pharmaceutical composition exclusive of enteric coating itself). In some embodiments, it adds less than 20%, especially from 12% to 20% to the weight of the uncoated composition. The enteric coating should be sufficient to prevent breakdown of the pharmaceutical composition of the present technology in 0.1N HCl for at least two hours, then capable of permitting complete release of all contents of the pharmaceutical composition within thirty minutes after pH is increased to 6.3 in a dissolution bath in which the composition is rotating at 100 revolutions per minute.

### E. Other Embodiments

In some embodiments, the weight ratio of pH-lowering agent(s) to absorption enhancer(s) is 3:1 to 20:1, 4:1 to 12:1, or 5:1 to 10:1. The total weight of all pH-lowering agents and the total weight of all absorption enhancers in a given pharmaceutical composition is included in the foregoing ratios. For example, if a pharmaceutical composition includes two pH-lowering agents and three absorption enhancers, the foregoing ratios will be computed on the total combined weight of both pH-lowering agents and the total combined weight of all three absorption enhancers.

Typically, the pH-lowering agent, the aromatic-cationic peptide and the absorption enhancer (whether single compounds or a plurality of compounds in each category) should be uniformly dispersed in the finished pharmaceutical product. In one embodiment, the finished pharmaceutical product may be produced in the form of a laminate having two or more layers, wherein the aromatic-cationic peptide is contained within a first layer and the pH-lowering agent and absorption enhancer are contained within a second layer laminated with the first layer. In another embodiment, the composition of the product comprises granules that include a pharmaceutical binder having the aromatic-cationic peptide, the pH-lowering agent and the absorption enhancer uniformly dispersed within the binder. Granules may also consist of an acid core, surrounded by a uniform layer of organic acid, a layer of enhancer and a layer of peptide that is surrounded by an outer layer of organic acid. Granules may be prepared from an aqueous mixture consisting of pharmaceutical binders such as polyvinyl pyrrolidone or hydroxypropyl methylcellulose, together with the pH-lowering agents, absorption enhancers and aromatic-cationic peptides of the present technology used in the present formulations.

### F. Manufacturing Process

In some embodiments, the present formulations are manufactured as follows:

The dosage form of the present formulations comprise, in a some embodiments, a tablet comprising a lamination of at least two layers. As used herein, the term “lamination” shall have its conventional meaning as something which is composed of layers of firmly united material, but which involves little, if any, interaction between the layers. The primary component of the first layer is typically the pH-lowering agent described above. The primary components of the second layer are typically the aromatic-cationic peptide (or a pharmaceutically acceptable salt thereof, such as acetate salt or trifluoroacetate salt) and the absorption enhancer. When combined in the manner described below, the constituents form a tablet having at least two layers. The layers may lie adjacent one another, e.g., the first layer on the top of the finished pharmaceutical product with the second layer being on the bottom or alternately, the first layer may lie within and thereby be encompassed by, the second layer. Although a two layer tablet is convenient to manufacture, it is also possible to have three or more layers wherein the second layer is substantially comprised of the peptide and the third layer comprises the surfactant.

The first layer is manufactured by granulating at least one pH-lowering agent to form a first layer material. While citric acid may be used as pH-lowering agent, citric acid alone typically does not exhibit the required compressibility characteristics. Therefore, during and after the granulation, other materials may be added to the pH-lowering agent to improve its mechanical properties. Specifically, during granulation in a fluidized bed, filler materials such as microcrystalline cellulose and a povidone binder may be added in amounts well known in the art. Next, the resultant granulation is dried and optionally sized in a mill in any manner well understood to those of ordinary skill in the art. Additionally, the granulation may be combined with glidants and lubricants such as talc and magnesium stearate, as described above, to farther improve compressibility and flowability of the granulation, thereby forming the first layer material.

The second layer material is formed by combining a peptide and at least one absorption enhancer (i.e., a surfactant). The second layer also may be manufactured in a fluidized bed. Because the peptide exhibits relatively high biological activity in small quantities, the second layer is produced by spraying the aromatic-cationic peptide and a binding agent, such as povidone, upon a surfactant or a mixture of at least one excipient and the surfactant. As described above, the surfactant is typically an acyl-carnitine, with lauroyl 1-carnitine in the present formulations. The optional excipient typically comprises an amount of a filler, such as microcrystalline cellulose, sufficient to provide proper adhesion between the layers, as understood by one of ordinary skill in the art. The resultant granulation is then dried and optionally sized in a mill in any manner well understood to those of ordinary skill in the art. Finally, the granulation is optionally transferred to a blender where the granulation is optionally blended with a disintegrant such as croscarmellose sodium or one or more other suitable disintegrants in amounts up to about 10.0% of the weight of the granulation, with about 2.0% by weight optimal. Although optional, disintegrants are believed to enhance bioavailability of the peptide by facilitating more complete release of the aromatic-cationic peptide near the same time as the release of the pH-lowering agent.

Other lubricants and additives such as magnesium stearate and stearic acid as well as other excipients such as colloidal silicon dioxide and povidone may also be added to improve the properties of the second layer material in a manner known in the art.

Next, a portion of the first layer material is fed to a standard two-layer tableting press and filled into a die or mold. The first layer material is then partially compressed to create a first layer. The partial compression is typically necessary to prevent substantial mixing between the first layer material and the second layer material when the second layer material is added to the die. Subsequent to partial compression of the first layer material, the second layer material is then added to the die containing the first layer. The first and second layer materials are then compressed together to form a tablet having two layers.

Typically, the first layer material constitutes about 50% to 90% of the total weight of the final tablet. Optimally, the first layer material constitutes about 70% of the total weight of the tablet. The second layer material typically constitutes about 50% to 10% of the total weight of the final tablet. Optimally, the second layer material comprises about 30% of the total weight of the final tablet.

Since the first layer material had been previously partially compressed into a layer, substantial mixing of the second layer material with the first layer material is avoided. The two layer structure of the present formulations substantially prevents contact between the pH-lowering agent and the peptide and surfactant. Specifically, at the interface between the two layers, typically less than 0.1% of the aromatic-cationic peptide of the present technology contacts the pH-lowering agent.

In an alternate embodiment, the finished pharmaceutical product of the present technology may include a size 00 gelatin capsule filled with 0.001 mg to about 1 mg of aromatic-cationic peptide, from about 0.0.1 mg to about 0.5 mg, or about 0.25 mg of aromatic-cationic peptide, 400 mg of granular citric acid (available, for example, from Archer Daniels Midland Corp.), 50 mg of taurodeoxycholic acid (available, for example, from SIGMA) and 50 mg lauroyl carnitine (SIGMA). All of the ingredients are adapted for eventual insertion into the gelatin capsule, and are optionally powders which may be added to a blender in any order. Thereafter, in some embodiments, the blender is run for about 5 minutes until the powders are thoroughly intermixed. Then the mixed powders are loaded into the large end of the gelatin capsules. The other end of the capsule is then added, and the capsules are snapped shut.

Because of the enhanced bioavailability provided by the present formulations, the concentration of the aromatic-cationic peptide (e.g., aromatic-cationic peptides of the present technology such as Phe-D-Arg-Phe-Lys-NH₂ and D-Arg-2′6′-Dmt-Lys-Phe-NH₂, calcitonin, PTH, Vasopressin, DALDA, DMT-DALDA, insulin, etc.) in the pharmaceutical preparation of the present technology may be kept relatively low. Specific formulation examples are set forth in the examples provided infra.

In some embodiments, the present formulations are manufactured as follows:

In some embodiments, a pharmaceutical composition of the present technology includes a size OO gelatin or HPMC (hydroxypropylmethyl cellulose) capsule filled with 0.25 mg of aromatic-cationic peptide, 400 mg of granular citric acid (available for example from Archer Daniels Midland Corp.) and 50 mg lauroyl carnitine (SIGMA)

All of the ingredients are for eventual insertion into the gelatin or HPMC capsule, and are powders which may be added to a blender in any order. Thereafter, the blender is run for about five minutes until the powders are thoroughly intermixed. Then the mixed powders are loaded into the large end of the gelatine capsules. The other end of the capsule is then added, and the capsule snapped shut. 500 or more such capsules may be added to a coating device (e.g., Vector LDCS 20/30 Laboratory Development Coating System (available from Vector Corp., Marion, Iowa)).

An enteric coating solution is made as follows. Weigh 500 grams of EUDRAGIT L30 D-55 (a methacrylic acid copolymer with methacylic acid methyl ester, an enteric coating available from ROHM Pharma Polymers Inc., Maidan, Mass.). Add 411 grams distilled water, 15 grams triethyl citrate and 38 grams talc. This amount of coating will be sufficient to coat about 500 size 00 capsules.

The capsules are weighed and placed into the drum of the coating machine. The machine is turned on to rotate the drum (now containing capsules) at 24-28 rpm. The temperature of inlet sprayer may be about 45° C. Exhaust temperatures may be about 30° C. Uncoated capsule temperature may be about 25° C. Air flow may be about 38 cubic feet per minute.

A tube from the machine is then inserted into the coating solution prepared as discussed above. The pump is then turned on for feeding solution into the coating device. Coating then proceeds automatically. The machine can be stopped at any time to weigh capsules to determine if the coating amount is sufficient. Usually coating is allowed to proceed for 60 minutes. The pump is then turned off for about five minutes while the machine is still running to help dry the coated capsules. The machine can then be turned off. The capsule coating is then complete, although it is recommended that the capsules be air dried for about two days.

Because of the enhanced bioavailability provided by the present technology, the concentration of the aromatic-cationic peptide component in the pharmaceutical preparation of the present disclosure may be kept relatively low. Specific formulation examples incorporating aromatic-cationic peptides of the present technology are set forth infra.

### III. Oral Delivery of Peptides Using Enzyme-Cleavable Translocators

In accordance with the disclosed methods, patients in need of treatment with aromatic-cationic peptides of the present technology are provided with an oral pharmaceutical composition thereof. In some embodiments, the composition is in the form of a tablet or capsule form of an ordinary size in the pharmaceutical industry. The dosages and frequency of administering the products are discussed in more detail below. Patients who may benefit are any who suffer from disorders that respond favorably to increased levels of a peptide-containing compound.

Aromatic-cationic peptides of the present technology display higher bioavailability when administered orally in accordance with the present methods compared to controls. In an oral formulation, the bioavailability of aromatic-cationic peptides of the present technology when linked to a membrane translocator (MT) according to the methods disclosed herein is significantly increased.

Without intending to be bound by theory, the pharmaceutical composition of the present disclosure is believed to overcome a series of different and unrelated natural barriers to bioavailability. Various components of the pharmaceutical compositions act to overcome different barriers by mechanisms appropriate to each, and result in synergistic effects on the bioavailability of a peptide active ingredient.

The aromatic-cationic peptide may be administered orally. In accordance with the methods, the presence of at least one MT, or at least two MTs, to enhance the membrane permeability of the fusion peptide across the lumen of the intestine and provide for improved bioavailability. Since the MT link to the active peptide can be cleaved by an enzyme in the blood or the lymphatic system, thereby leaving the active peptide free to reach its target.

Also, in accordance with the method, proteolytic degradation of the peptide and of the membrane translocator by stomach enzymes (most of which are active in the acid pH range) and intestinal or pancreatic proteases (most of which are active in the neutral to basic pH range) is reduced.

Again, without intending to be bound by theory, it appears that, in accordance with the present method, the peptide is transported through the stomach under the protection of an appropriate acid-resistant protective vehicle for substantially preventing contact between the aromatic-cationic peptide or other peptide and any stomach proteases capable of degrading it. Once the pharmaceutical composition passes through the stomach and enters the intestinal region where basic to neutral pH predominates, and where proteases tend to have basic to neutral pH optima, the enteric coating or other vehicle releases the peptide and acid or protease inhibitors (in close proximity to each other).

The acid is believed to lower the local intestinal pH, where the aromatic-cationic peptide has been released, to levels below the optimal range for many intestinal proteases and other intestinal enzymes. This decrease in pH reduces the proteolytic activity of the intestinal proteases, thus affording protection to the peptide and the membrane translocator from potential degradation. The activity of these proteases is diminished by the temporarily acidic environment provided by the composition. According to the methods, sufficient acid is provided that local intestinal pH is lowered temporarily to 5.5 or below, 4.7 or below, or 3.5 or below. The sodium bicarbonate test described below (in the section captioned “the pH-Lowering Agent”) is indicative of the required acid amount. Conditions of reduced intestinal pH persist for a time period sufficient to protect the aromatic-cationic peptide and the membrane translocator from proteolytic degradation until at least some of the aromatic-cationic peptide has had an opportunity to cross the intestinal wall into the bloodstream. For salmon calcitonin, experiments have demonstrated a Tₘₐₓ of 5-15 minutes for blood levels of salmon calcitonin when the active components are injected directly into the duodenum, ileum or colon of rats.

Alternatively, protease inhibitors are believed to reduce the proteolytic activity of the intestinal proteases, thus affording protection to the peptide and the membrane translocator from premature potential degradation.

Compositions of the present disclosure can optionally contain absorption enhancers. The absorption enhancers of the disclosure synergistically promote peptide absorption into the blood while conditions of reduced proteolytic activity prevail.

The mechanism by which the method is believed to accomplish the goal of enhanced bioavailability is aided by having active components of the pharmaceutical composition released together as simultaneously as possible. According to the methods, the volume of enteric coating is kept as low as possible consistent with providing protection from stomach proteases. Thus enteric coating is less likely to interfere with peptide release, or with the release of other components in close time proximity with the peptide. The enteric coating should normally add less than 30% to the weight of the remainder of pharmaceutical composition (i.e., the other components of the composition excluding enteric coating). In some embodiments, it is less than 20%. In some embodiments, the enteric coating adds between 10% and 20% to the weight of the uncoated ingredients.

The absorption enhancer which may be a solubility enhancer and/or transport enhancer (as described in more detail below) aids transport of the aromatic-cationic peptide from the intestine to the blood, and may promote the process so that it better occurs during the time period of reduced intestinal pH and reduced intestinal proteolytic activity. Many surface agents may act as both solubility enhancers and transport (uptake) enhancers. Again without intending to be bound by theory, it is believed that enhancing solubility provides (1) a more simultaneous release of the active components of the present methods into the aqueous portion of the intestine, (2) better solubility of the peptide in, and transport through, a mucous layer along the intestinal walls. Once the peptide active ingredient reaches the intestinal walls, an uptake enhancer provides better transport through the brush border membrane of the intestine into the blood, via either transcellular or paracellular transport. As discussed in more detail below, some compounds may provide both functions. In those instances, embodiments utilizing both of these functions may do so by adding only one additional compound to the pharmaceutical composition. In other embodiments, separate absorption enhancers may provide the two functions separately.

Each of the ingredients of the pharmaceutical composition of the present disclosure is separately discussed below. Combinations of multiple pH-lowering agents, or multiple enhancers can be used as well as using just a single pH-lowering agent and/or single enhancer.

### A. Peptide Active Ingredients

Peptide active ingredients which may benefit from oral delivery in accordance with the methods include any therapeutic agent that is physiologically active and has a plurality of amino acids and at least one peptide bond in its molecular structure. These peptide active ingredients are linked to an MT sequence to facilitate their absorption from the intestine. The MT must be protected from cleavage by proteases in the stomach and intestine before its absorption. However, once absorbed, the MT should be able to be at least partially removed by proteases to free up the active peptide.

The MT can comprise an amino acid sequence, such as a signal peptide or signal sequence. A “signal peptide,” as used herein, is a sequence of amino acids generally but not necessarily of a length of about 10 to about 50 or more amino acid residues, many (typically about 55-60%) residues of which are hydrophobic such that they have a hydrophobic, lipid-soluble portion. The hydrophobic portion is a common, major motif of the signal peptide, and it is often a central part of the signal peptide of protein secreted from cells. A signal peptide is a peptide capable of penetrating through the cell membrane to allow the export of cellular proteins. The signal peptides of this method, as discovered herein, are also “importation competent,” i.e., capable of penetrating through the cell membrane from outside the cell to the interior of the cell. The amino acid residues can be mutated and/or modified (i.e., to form mimetics) so long as the modifications do not affect the translocation-mediating function of the peptide. Thus the word “peptide” includes mimetics and the word “amino acid” includes modified amino acids, as used herein, unusual amino acids, and D-form amino acids. All importation competent signal peptides encompassed by this method have the function of mediating translocation across a cell membrane from outside the cell to the interior of the cell. They may also retain their ability to allow the export of a protein from the cell into the external milieu. A putative signal peptide can easily be tested for this importation activity following the teachings provided herein, including testing for specificity for any selected cell type.

Table 1 exemplifies amino acid sequences, each of which can be used as an MT.

TABLE 1
Amino Acid Sequences of Some
MT Peptides and Their Sources

| SEQUENCE | SEQUENCE DERIVATION | SOURCE |
|---|---|---|
| ALA-ALA-VAL- | Signal Peptide from | U.S. Pat. No. |
| ALA-LEU- | Kaposi Fibroblast | 5,807,746 |
| LEU-PRO-ALA- | Growth Factor | |
| VAL-LEU-LEU- | | |
| ALA-LEU-LEU- | | |
| ALA-PRO-VAL- | | |
| ASN-ARG-LYS- | | |
| ARG-ASN-LYS- | | |
| LEU-MET-PRO | | |
| (SEQ ID No. 1) | | |
| TYR-GLY-ARG- | Protein | Schwarz et al. |
| LYS-LYS-ARG- | Transduction Domain | (1999), Science |
| ARG-GLN-ARG- | of HIV TAT Protein | 285:1569 |
| ARG-ARG | | |
| (SEQ ID No. 2) | | |
| VAL-THR-VAL- | Signal Sequence of | Zhang et al. |
| LEU-ALA-LEU- | Human Integrin β₃ | (1988) PNAS 95: |
| GLY-ALA-LEU- | | 9184 |
| ALA-GLY-VAL- | | |
| GLY-VAL-GLY | | |
| (SEQ ID No. 3) | | |
| 38 kDa Protein | HSV-VP22 Protein | Phelan at al. |
| | | (1998), Nature |
| | | Biotechnology |
| | | 16:440 |
| ALA-ALA-VAL- | Modified from 16- | Rojas et al |
| LEU-LEU-PRO- | residue hydrophobic | (1998) Nature |
| VAL-LEU-LEU- | region of signal | Biotechnology |
| ALA-ALA-PRO | sequence of Kaposi | 16:370 |
| (SEQ ID No. 4) | fibroblast growth | |
| | factor. | |

The MT can also comprise fatty acids and/or bile acids. Such molecules, when used, are linked to the active peptide by an amino acid bridge which is subject to cleavage by proteases in the plasma. Alternatively, the MT can be linked to the active peptide by a non-peptidyl linkage, in which case the in vivo enzyme that cleaves the linkage may be an enzyme other than protease. The amino acid bridge must be a target for cleavage by at least one plasma protease. Plasma proteases as well as their target sequences are well known in the art. Table 2 illustrates some of these enzymes as well as their specific targets

TABLE 2
Plasma Proteases and their Specific Targets

| PROTEASE | SPECIFIC TARGET | REMARKS |
|---|---|---|
| Caspase-1 | Tyr-Val-Ala-Asp-Xaa* | |
| | (SEQ ID No. 5) | |
| Caspase-3 | Asp-Xaa-Xaa-Asp-Xaa | |
| | (SEQ ID No. 6) | |
| Proprotein | Arg-(Xaa)ₙ-Arg-Xaa | n = 2, 4 or 6 |
| convertase 1 | (SEQ ID No. 7) | |
| | Lys-(Xaa)ₙ-Arg-Xaa | n = 2, 4, or 6 |
| | (SEQ ID No. 8) | |
| | Arg-Arg-Xaa | |
| | Lys-Arg-Xaa | |
| Proprotein | same as proprotein | |
| convertase 2 | convertase 1 | |
| Proprotein | Glp-Arg-Thr-Lys-Arg- | |
| convertase 4 | Xaa (SEQ ID No. 9) | |
| Proprotein | Arg-Val-Arg-Arg-Xaa | |
| convertase | (SEQ ID No. 10) | |
| 4 PACE 4 | Decanoyl-Arg-Val- | |
| | Arg-Arg-Xaa (SEQ ID | |
| | No. 11) | |
| Prolyl oligopeptidase | Pro-Xaa | |
| Endothelin cleaving | Trp-Val-Pro-Xaa (SEQ | |
| enzyme followed by | ID No. 12) | |
| dipeptidyl-peptidase | Trp-Val-Ala-Xaa | |
| IV | (SEQ ID No. 13) | |
| Signal peptidase | | depends on nearby |
| | | amino acid |
| Neprilysin followed | Xaa-Phe-Yaa-Xaa | broad specificity, |
| by dipeptidyl- | (SEQ ID No. 14) | max length =40 |
| peptidase IV | | amino acids |
| | Xaa-Tyr-Yaa-Xaa | |
| | (SEQ ID No. 15) | |
| | Xaa-Trp-Yaa-Xaa | |
| | SEQ ID No. 16) | |
| Renin followed by | Asp-Arg-Tyr-Ile-Pro- | substitute Pro or Ala |
| dipeptidyl-peptidase | Phe-His-Leu-Leu-Val- | for Val & Ser |
| IV | Tyr-Ser (SEQ ID No. | |
| | 17) | |

*The N-terminal side of bolded amino acids is the specific target for the protease cleavage.

The method, by several mechanisms, suppresses the degradation of the active ingredient linked to an MT by protease that would otherwise tend to cleave one or more of the peptide bonds of the active ingredient. The molecular structure of the active ingredient may further include other substituents or modifications. For example, aromatic-cationic peptides of the present technology can be amidated at the C-terminus. Both synthetic and natural peptides can be orally delivered in accordance with the method.

Peptide active compounds of the present disclosure include, but are not limited to, aromatic-cationic peptides of the present technology, as well as polypeptides such as insulin, vasopressin, and calcitonin. Other examples include calcitonin gene-related peptide, parathyroid hormone, luteinizing hormone-releasing factor, erythropoietin, tissue plasminogen activators, human growth hormone, adrenocorticototropin, various interleukins, enkephalin, glucagon-like peptide 1, and all analogs thereof. Many others are known in the art. It is expected that any pharmaceutical compound having peptide bonds which would be subject to cleavage in the gastrointestinal tract would benefit from oral delivery in accordance with the present methods because of the enhancement of absorption of such compounds from the intestine coupled with the reduction in such cleavage that is afforded by the present methods.

When aromatic-cationic peptides of the present technology are used, they may comprise from 0.02 to 0.2 percent by weight relative to the total weight of the overall pharmaceutical composition (exclusive of enteric coating). Other peptide peptides may be present at higher or lower concentrations depending on desired target blood concentrations for the active compound and its bioavailability in the oral delivery system of the methods.

Aromatic-cationic peptides of the present technology may be made by either chemical or recombinant syntheses known in the art. Precursors of other amidated peptides may be made in like manner. Recombinant production is believed to be significantly more cost effective. For example, enzymatic amidation is described in U.S. Pat. No. 4,708,934 and European Patent Publications 0 308 067 and 0 382 403. Recombinant production may be used for both the precursor and the enzyme that catalyzes the conversion of the precursor to the final product. Such recombinant production is discussed in Biotechnology, Vol. 11 (1993) pp. 64-70, which further describes a conversion of a precursor to an amidated product.

The linking of an MT to an active peptide ingredient may also be made by either chemical or recombinant syntheses known in the art. By “linking” as used herein is meant that the biologically active peptide is associated with the MT in such a manner that when the MT crosses the cell membrane, the active peptide is also imported across the cell membrane. Examples of such means of linking include (A) linking the MT to the active peptide by a peptide bond, i.e., the two peptides (the peptide part of the MT and the active peptide) can be synthesized contiguously; (B) linking the MT to the active peptide by a non-peptide covalent bond (such as conjugating a signal peptide to a protein with a crosslinking reagent); (C) chemical ligation methods can be employed to create a covalent bond between the carboxy-terminal amino acid of an MT such as a signal peptide and the active peptide.

Examples of method (A) are shown below wherein a peptide is synthesized, by standard means known in the art, (Merrifield, J. Am. Chem. Soc. 85:2149-2154, 1963; and Lin et al., Biochemistry 27:5640-5645, 1988) and contains, in linear order from the amino-terminal end, a signal peptide sequence (the MT), an amino acid sequence that can be cleaved by a plasma protease, and a biologically active amino acid sequence. Such a peptide could also be produced through recombinant DNA techniques, expressed from a recombinant construct encoding the above-described amino acids to create the peptide. (Sambrook et al., Molecular Cloning: A Laboratory Manual, 2nd Ed., Cold Spring Harbor Laboratory, Cold Spring Harbor, N.Y., 1989).

For method (B), either a peptide bond, as above, can be utilized or a non-peptide covalent bond can be used to link the MT with the biologically active peptide, polypeptide or protein. This non-peptide covalent bond can be formed by methods standard in the art, such as by conjugating the MT to the peptide, polypeptide or protein via a crosslinking reagent, for example, glutaraldehyde. Such methods are standard in the art. (Walter et al., Proc. Natl. Acad. Sci. USA 77:5197; 1980).

For method (C), standard chemical ligation methods, such as using chemical crosslinkers interacting with the carboxy-terminal amino acid of a signal peptide, can be utilized. Such methods are standard in the art (Goodfriend et al., Science 143:1344; 1964, which uses water-soluble carbodiimide as a ligating reagent) and can readily be performed to link the carboxy terminal end of the signal peptide to any selected biologically active molecule.

### B. The pH-Lowering Agent and Protease Inhibitor

The total amount of the pH-lowering compound to be administered with each administration of aromatic-cationic peptide may be an amount which, when it is released into the intestine, is sufficient to lower the local intestinal pH substantially below the pH optima for proteases found there. The quantity required will necessarily vary with several factors including the type of pH-lowering agent used (discussed below) and the equivalents of protons provided by a given pH-lowering agent. In practice, the amount required to provide good bioavailability is an amount which, when added to a solution of 10 milliliters of 0.1 M sodium bicarbonate, lowers the pH of that sodium bicarbonate solution to no higher than 5.5, no higher than 4.7, or no higher than 3.5. Enough acid to lower pH, in the foregoing test, to about 2.8 may been used in some embodiments. In some embodiments at least 300 milligrams, or at least 400 milligrams of the pH-lowering agent is used in the pharmaceutical composition of the methods. The foregoing values relate to the total combined weight of all pH-lowering agents where two or more of such agents are used in combination. The oral formulation should not include an amount of any base which, when released together with the pH-lowering compound, would prevent the pH of the above-described sodium bicarbonate test from dropping to 5.5 or below.

The pH-lowering agent of the methods may be any pharmaceutically acceptable compound that is not toxic in the gastrointestinal tract and is capable of either delivering hydrogen ions (a traditional acid) or of inducing higher hydrogen ion content from the local environment. It may also be any combination of such compounds. In some embodiments, at least one pH-lowering agent used in the methods have a pKa no higher than 4.2, or no higher than 3.0. In some embodiments, the pH lowering agent has a solubility in water of at least 30 grams per 100 milliliters of water at room temperature.

Examples of compounds that induce higher hydrogen ion content include aluminum chloride and zinc chloride. Pharmaceutically acceptable traditional acids include, but are not limited to acid salts of amino acids (e.g. amino acid hydrochlorides) or derivatives thereof. Examples of these are acid salts of acetylglutamic acid, alanine, arginine, asparagine, aspartic acid, betaine, carnitine, carnosine, citrulline, creatine, glutamic acid, glycine, histidine, hydroxylysine, hydroxyproline, hypotaurine, isoleucine, leucine, lysine, methylhistidine, norleucine, ornithine, phenylalanine, proline, sarcosine, serine, taurine, threonine, tryptophan, tyrosine and valine. In some embodiments, the pharmaceutical formulation comprises acetate salt or trifluoroacetate salt.

Other examples of useful pH-lowering compounds include carboxylic acids such as acetylsalicylic, acetic, ascorbic, citric, fumaric, glucuronic, glutaric, glyceric, glycocolic, glyoxylic, isocitric, isovaleric, lactic, maleic, oxaloacetic, oxalosuccinic, propionic, pyruvic, succinic, tartaric, valeric, and the like.

Other useful pH-lowering agents that might not usually be called “acids” in the art, but which may nonetheless be useful in accordance with the methods are phosphate esters (e.g., fructose 1,6 diphosphate, glucose 1,6 diphosphate, phosphoglyceric acid, and diphosphoglyceric acid). CARBOPOL (Trademark BF Goodrich) and polymers such as polycarbophil may also be used to lower pH.

Any combination of pH lowering agent that achieves the required pH level of no higher than 5.5 in the sodium bicarbonate test discussed above may be used. One embodiment utilizes, as at least one of the pH-lowering agents of the pharmaceutical composition, an acid selected from the group consisting of citric acid, tartaric acid and an acid salt of an amino acid.

When aromatic-cationic peptides of the present technology are used, certain ratios of pH-lowering agent to peptide have proven especially effective. In some embodiments, the weight ratio of pH-lowering agent to aromatic-cationic peptide of the present technology exceed 200:1, 800:1, or 2000:1.

An alternative or a supplement to the use of pH-lowering agents is the use of protease inhibitors, in particular inhibitors of intestinal proteases. Table 3 illustrates some of the known intestinal proteases.

TABLE 3
Intestinal Proteases and their Specific Targets

| | TARGET | pH | |
|---|---|---|---|
| PROTEASE | SITE | OPTIMUM | REMARKS |
| Trypsin | Lys-Xaa | 8 | |
| | Arg-Xaa | | |
| Chymotrypsin | Tyr-Xaa | 7.0-9.0 | |
| | Phe-Xaa | | |
| | Trp-Xaa | | |
| Elastase | Ala-Xaa | 8.8 | |
| | Val-Xaa | | |
| | Leu-Xaa | | |
| | Ile-Xaa | | |
| | Gly-Xaa | | |
| | Ser-Xaa | | |
| Kallikrein | Arg-Xaa | 7.0-8.0 | |
| | Phe-Arg-Xaa | | preferred |
| | Leu-Arg-Xaa | | preferred |
| Carboxypeptidase | Xaa-Xaa | 7.0-9.0 | from C-terminal |

### C. Optional Absorption Enhancer

When used, absorption enhancers may be present in a quantity that constitutes from 0.1 to 20.0 percent by weight, relative to the overall weight of the pharmaceutical composition (exclusive of the enteric coating). Illustrative absorption enhancers are surface active agents which act both as solubility enhancers and uptake enhancers. Generically speaking, “solubility enhancers” improve the ability of the components of the methods to be solubilized in either the aqueous environment into which they are originally released or into the lipophilic environment of the mucous layer lining the intestinal walls, or both. “Transport (uptake) enhancers” (which are frequently the same surface active agents used as solubility enhancers) are those which facilitate the ease by which aromatic-cationic peptides of the present technology cross the intestinal wall.

One or more absorption enhancers may perform one function only (e.g., solubility), or one or more absorption enhancers may perform the other function only (e.g., uptake), within the scope of the methods. It is also possible to have a mixture of several compounds some of which provide improved solubility, some of which provide improved uptake and/or some of which perform both. Without intending to be bound by theory, it is believed that uptake enhancers may act by (1) increasing disorder of the hydrophobic region of the membrane exterior of intestinal cells, allowing for increased transcellular transport; or (2) leaching membrane proteins resulting in increased transcellular transport; or (3) widening pore radius between cells for increased paracellular transport.

Surface active agents are believed to be useful both as solubility enhancers and as uptake enhancers. For example, detergents are useful in (1) solubilizing all of the active components quickly into the aqueous environment where they are originally released, (2) enhancing lipophilicity of the components of the methods, especially the aromatic-cationic peptide, aiding its passage into and through the intestinal mucus, (3) enhancing the ability of the normally polar aromatic-cationic peptide to cross the epithelial barrier of the brush border membrane; and (4) increasing transcellular or paracellular transport as described above.

When surface active agents are used as the absorption enhancers, they may be free flowing powders for facilitating the mixing and loading of capsules during the manufacturing process. Because of inherent characteristics of aromatic-cationic peptide of the present technology and other peptides (e.g., their isoelectric point, molecular weight, amino acid composition, etc.), certain surface active agents interact best with certain peptides. Indeed, some can undesirably interact with the charged portions of aromatic-cationic peptide of the present technology and prevent its absorption, thus undesirably resulting in decreased bioavailability. When trying to increase the bioavailability of aromatic-cationic peptides of the present technology or other peptides, absorption enhancers may be selected from the group consisting of (i) anionic surface active agents that are cholesterol derivatives (e.g., bile acids), (ii) cationic surface agents (e.g., acyl carnitines, phospholipids and the like), (iii) non-ionic surface active agents, and (iv) mixtures of anionic surface active agents (especially those having linear hydrocarbon regions) together with negative charge neutralizers. Negative charge neutralizers include but are not limited to acyl carnitines, cetyl pyridinium chloride, and the like. The absorption enhancer may be soluble at acid pH, particularly in the 3.0 to 5.0 range.

One embodiment uses a mixture of cationic surface active agents and anionic surface active agents that are cholesterol derivatives, both of which are soluble at acid pH.

One embodiment uses an acid soluble bile acid together with a cationic surface active agent. An acyl carnitine and sucrose ester is a good combination. When a particular absorption enhancer is used alone, it may be a cationic surface active agent. Acyl carnitines (e.g., lauroyl carnitine), phospholipids and bile acids are particularly good absorption enhancers, especially acyl carnitine. Anionic surfactants that are cholesterol derivatives are also used in some embodiments. According to the present methods, aromatic-cationic peptide that interfere with its absorption into the blood are avoided.

To reduce the likelihood of side effects, detergents used as absorption enhancers are either biodegradable or reabsorbable (e.g. biologically recyclable compounds such as bile acids, phospholipids, and/or acyl carnitines). Acylcarnitines are believed particularly useful in enhancing paracellular transport. When a bile acid (or another anionic detergent lacking linear hydrocarbons) is used in combination with a cationic detergent, aromatic-cationic peptides of the present technology are better transported both to and through the intestinal wall.

Illustrative absorption enhancers include but are not limited to: (a) salicylates such as sodium salicylate, 3-methoxysalicylate, 5-methoxysalicylate and homovanilate; (b) bile acids such as taurocholic, tauorodeoxycholic, deoxycholic, cholic, glycholic, lithocholate, chenodeoxycholic, ursodeoxycholic, ursocholic, dehydrocholic, fusidic, etc.; (c) non-ionic surfactants such as polyoxyethylene ethers (e.g. Brij 36T, Brij 52, Brij 56, Brij 76, Brij 96, Texaphor A6, Texaphor A14, Texaphor A60 etc.), p-t-octyl phenol polyoxyethylenes (Triton X-45, Triton X-100, Triton X-114, Triton X-305 etc.) nonylphenoxypoloxyethylenes (e.g. Igepal CO series), polyoxyethylene sorbitan esters (e.g. Tween-20, Tween-80 etc.); (d) anionic surfactants such as dioctyl sodium sulfosuccinate; (e) lyso-phospholipids such as lysolecithin and lysophosphatidylethanolamine; (f) acylcarnitines, acylcholines and acyl amino acids such as lauroylcarnitine, myristoylcarnitine, palmitoylcarnitine, lauroylcholine, myristoylcholine, palmitoylcholine, hexadecyllysine, N-acylphenylalanine, N-acylglycine etc.; g) water soluble phospholipids; (h) medium-chain glycerides which are mixtures of mono-, di- and triglycerides containing medium-chain-length fatty acids (caprylic, capric and lauric acids); (i) ethylene-diaminetetraacetic acid; (j) cationic surfactants such as cetylpyridinium chloride; (k) fatty acid derivatives of polyethylene glycol such as Labrasol, Labrafac, etc.; and (l) alkylsaccharides such as lauryl maltoside, lauroyl sucrose, myristoyl sucrose, palmitoyl sucrose, etc.

In some embodiments, cationic ion exchange agents (e.g. detergents) are included to provide solubility enhancement by another possible mechanism. In particular, they may prevent the binding of aromatic-cationic peptides of the present technology or other peptides to mucus. Illustrative cationic ion exchange agents include but are not limited to protamine chloride or any other polycation.

### D. Other Optional Ingredients

In some embodiments, a water-soluble barrier separates the protease inhibitors and/or the pH-lowering agent from the acid resistant protective vehicle. A conventional pharmaceutical capsule can be used for the purpose of providing this barrier. Many water soluble barriers are known in the art and include, but are not limited to, hydroxypropyl methylcellulose and conventional pharmaceutical gelatins.

In some embodiments, another peptide (such as albumin, casein, soy protein, other animal or vegetable proteins and the like) is included to reduce non-specific adsorption (e.g., binding of peptide to the intestinal mucus barrier) thereby lowering the necessary concentration of the aromatic-cationic peptide. When added, the peptide may be comprise from 1.0 to 10.0 percent by weight relative to the weight of the overall pharmaceutical composition (excluding protective vehicle). This second peptide is not physiologically active and is may comprise a food peptide such as soy bean peptide or the like. Without intending to be bound by theory, this second peptide may also increase bioavailability by acting as a protease scavenger that desirably competes with the aromatic-cationic peptide for protease interaction. The second peptide may also aid the active compound's passage through the liver.

All pharmaceutical compositions of the present disclosure may optionally also include common pharmaceutical diluents, glidents, lubricants, gelatin capsules, preservatives, colorants and the like in their usual known sizes and amounts.

### E. The Protective Vehicle

Any carrier or vehicle that protects the aromatic-cationic peptide from stomach proteases and then dissolves so that the other ingredients of the methods may be released in the intestine is suitable. Many such enteric coatings are known in the art, and are useful in accordance with the methods. Examples include but are not limited to cellulose acetate phthalate, hydroxypropyl methylethylcellulose succinate, hydroxypropyl methylcellulose phthalate, carboxyl methylethylcellulose and methacrylic acid-methyl methacrylate copolymer. In some embodiments, the active peptide, absorption enhancers such as solubility and/or uptake enhancer(s), and pH-lowering compound(s), are included in a sufficiently viscous protective syrup to permit protected passage of the components of the methods through the stomach.

Suitable enteric coatings for protecting the aromatic-cationic peptide from stomach proteases may be applied, for example, to capsules after the remaining components of the compositions have been loaded within the capsule. In other embodiments, enteric coating is coated on the outside of a tablet or coated on the outer surface of particles of active components which are then pressed into tablet form, or loaded into a capsule, which is itself coated with an enteric coating.

It is very desirable that all components of the composition be released from the carrier or vehicle, and solubilized in the intestinal environment as simultaneously as possible. In some embodiments, the vehicle or carrier releases the active components in the small intestine where uptake enhancers that increase transcellular or paracellular transport are less likely to cause undesirable side effects than if the same uptake enhancers were later released in the colon. It is emphasized, however, that the present method is believed effective in the colon as well as in the small intestine. Numerous vehicles or carriers, in addition to the ones discussed above, are known in the art. It is desirable (especially in optimizing how simultaneously the components of the method are released) to keep the amount of enteric coating low. In some embodiments, the enteric coating adds no more than 30% to the weight of the remainder of pharmaceutical composition (the “remainder” being the pharmaceutical composition exclusive of enteric coating itself). In some embodiments, it adds less than 20%, especially from 12% to 20% to the weight of the uncoated composition. The enteric coating should be sufficient to prevent breakdown of the pharmaceutical composition of the methods in 0.1N HCl for at least two hours, then capable of permitting complete release of all contents of the pharmaceutical composition within thirty minutes after pH is increased to 6.3 in a dissolution bath in which the composition is rotating at 100 revolutions per minute.

### F. Other Embodiments

In some embodiments, weight ratio of pH-lowering agent(s) and/or protease inhibitors to absorption enhancer(s), when present, be between 3:1 and 20:1,4:1-12:1, or 5:1-10:1. The total weight of all pH-lowering agents and/or protease inhibitors and the total weight of all absorption enhancers in a given pharmaceutical composition is included in the foregoing ratios. For example, if a pharmaceutical composition includes two pH-lowering agents and three absorption enhancers, the foregoing ratios will be computed on the total combined weight of both pH-lowering agents and the total combined weight of all three absorption enhancers.

In some embodiments, the pH-lowering agent and/or protease inhibitor, the aromatic-cationic peptide and the absorption enhancer, when present, (whether single compounds or a plurality of compounds in each category) be uniformly dispersed in the pharmaceutical composition. In one embodiment, the pharmaceutical composition comprises granules that include a pharmaceutical binder having the aromatic-cationic peptide, the pH-lowering agent and the absorption enhancer uniformly dispersed within the binder. In some embodiments, granules may also consist of an acid core, surrounded by a uniform layer of organic acid, a layer of enhancer and a layer of peptide that is surrounded by an outer layer of organic acid. Granules may be prepared from an aqueous mixture consisting of pharmaceutical binders such as polyvinyl pyrrolidone or hydroxypropyl methylcellulose, together with the pH-lowering agents, absorption enhancers and aromatic-cationic peptide.

### G. Manufacturing Process

In some embodiments, the pharmaceutical composition of the present disclosure includes a size OO gelatin capsule filled with 0.25 mg. of aromatic-cationic peptides of the present technology linked to an MT, 400 mg. of granular citric acid (available for example from Archer Daniels Midland Corp.), 50 mg. of taurodeoxycholic acid (available for example from SIGMA), 50 mg. lauroyl carnitine (SIGMA).

All of the ingredients are for eventual insertion into the gelatin capsule, and may be powders which may be added to a blender in any order. Thereafter, the blender is run for about three minutes until the powders are thoroughly intermixed. Then the mixed powders are loaded into the large end of the gelatine capsules. The other end of the capsule is then added, and the capsule snapped shut. 500 or more such capsules may be added to a coating device (e.g., Vector LDCS 20/30 Laboratory Development Coating System (available from Vector Corp., Marion, Iowa)).

An enteric coating solution is made as follows. Weigh 500 grams of EUDRAGIT L30 D-55 (a methacrylic acid copolymer with methacylic acid methyl ester, an enteric coating available from RoHM Tech Inc., Maidan, Mass.). Add 411 grams distilled water, 15 grams triethyl citrate and 38 grams talc. This amount of coating will be sufficient to coat about 500 size OO capsules.

The capsules are weighed and placed into the drum of the coating machine. The machine is turned on to rotate the drum (now containing capsules) at 24-28 rpm. The temperature of inlet sprayer may be about 45° C. Exhaust temperatures may be about 30° C. Uncoated capsule temperature may be about 25° C. Air flow may be about 38 cubic feet per minute.

A tube from the machine is then inserted into the coating solution prepared as discussed above. The pump is then turned on for feeding solution into the coating device. Coating then proceeds automatically. The machine can be stopped at any time to weigh capsules to determine if the coating amount is sufficient. Usually coating is allowed to proceed for 60 minutes. The pump is then turned off for about five minutes while the machine is still running to help dry the coated capsules. The machine can then be turned off. The capsule coating is then complete, although it is recommended that the capsules be air dried for about two days.

Because of the enhanced bioavailability provided by the present methods, the concentration of expensive aromatic-cationic peptide in the pharmaceutical preparation may be kept relatively low.

### H. Treatment of Patients

Aromatic-cationic peptides of the present technology may be chosen as an active ingredient for treatment of medical conditions and diseases as recited herein. Nasally administered aromatic-cationic peptide will be effective against medical conditions or diseases such as those described herein. Serum levels may be measured by HPLC or mass spectroscopy, according to methods known in the art. The attending physician may monitor patient response, aromatic-cationic peptide blood levels, or surrogate markers of disease, especially during the initial phase of treatment. The physician may then alter the dosage somewhat to account for individual patient metabolism and response.

The bioavailability achievable in accordance with the present methods permits oral delivery of aromatic-cationic peptide into the blood at the above-identified concentration levels while using only 10-1000 micrograms of aromatic-cationic peptides of the present technology, 10-400 micrograms, or between 10 and 200 micrograms.

In some embodiments, a single capsule be used at each administration because a single capsule can provide simultaneous release of the polypeptide, pH-lowering agent and absorption enhancers. This is highly desirable because the acid is best able to reduce undesirable proteolytic attack on the peptide when the acid is released in close time proximity to release of the peptide. Near simultaneous release is best achieved by administering all components of the methods as a single pill or capsule. However, the methods also include, for example, dividing the required amount of acid and enhancers, when used, among two or more capsules which may be administered together such that they together provide the necessary amount of all ingredients.

### IV. Nasal Delivery of Peptide Pharmaceutical Compositions

Peptide active ingredients which may benefit from nasal delivery in accordance with the methods include any therapeutic agent that is physiologically active and has, as part of its molecular structure, a plurality of amino acids and at least one peptide bond. In addition to natural amino acids, the amino acids may be D-amino acids or unnatural amino acids, some examples of which are discussed infra. The molecular structure may further include other substituents or modifications. For example, aromatic-cationic peptide is amidated at its C-terminus. Some peptides may be amidated at locations that are not amidated in nature, or may be otherwise modified.

Peptide active compounds of the methods include, but are not limited to, aromatic-cationic peptides of the present technology, as well as polypeptides such as insulin, vasopressin, calcitonin (including not only salmon calcitonin, but other calcitonins as well). Other examples include calcitonin gene-related peptide, parathyroid hormone (including amidated or unamidated truncates thereof such as PTH1-31-amide or PTH1-34-amide), desmopressin, luteinizing hormone-releasing factor, erythropoietin, tissue plasminogen activators, human growth hormone, adrenocorticototropin, various interleukins, enkephalin, and the like. Many others are known in the art.

Both man-made and natural peptides can be delivered nasally in accordance with the methods. Thus, the peptide active compound, in some embodiments, could be aromatic-cationic peptides of the present technology, glucagon-like peptide-1 (GLP-1), or analogs thereof, desmopressin (DDAVP), leuprolide, 2,6-dimethyltyrosine-D-arginine-phenylalanine-lysine amide (DMT-DALDA), peptidomimetics and the like.

The peptides for use in the methods may be in free form or in pharmaceutically acceptable salt or complex form, e.g., in pharmaceutically acceptable acid addition salt form. Such salts and complexes are known and tend to possess an equivalent degree of activity and tolerability to the free forms. Suitable acid addition salt forms for use in accordance with the methods include for example the hydrochlorides and acetates.

### A. Enhancement of Bioavailability

Enhancement of bioavailability is achieved with one or more classes of enhancers selected from fatty acids, sugar esters of fatty acids, acyl carnitines and citrates. Some embodiments use combinations thereof, except that acyl carnitines and fatty acids are not used together because of undesirable interaction between them. Molecular structures regarding each class is discussed below.

### B. Fatty Acids

Without intending to be bound by theory, it is believed that the fatty acids interact with peptides to desirably enhance their ability to penetrate cell membranes, thus enhancing transcellular transport. The hydrophobic region of fatty acids is believed important to this function, and should desirably include as many consecutive carbon atoms as possible, consistent with water solubility, at least 8 consecutive carbon atoms, or 10-14 carbon atoms. Illustrative fatty acids include but are not limited to lauric acid and oleic acid. When used, concentration of fatty acid, may be between 0.1 and 4.0 mg/mL, or between 0.5 and 2.0 mg/mL.

### C. Sugar Esters of Fatty Acids

Without intending to be bound by theory, it is believed that the sugar esters of fatty acids may interact with cells in a manner that could alter their shape, increase pore size, and thereby desirably increase paracellular transport. They may also provide benefit in transcellular transport. When fatty acids and sugar esters of fatty acids are used in combination, bioavailability may be especially enhanced by the combination of enhanced transcellular and enhanced paracellular transport. Like the fatty acids, the hydrophobic region may also include at least 8 consecutive carbon atoms, especially 10-14 carbon atoms. The sugar moiety may aid water solubility. Illustrative sugar esters of fatty acids include but are not limited to sucrose laurate, glucose laurate and fructose laurate. When used, concentration of sugar esters of fatty acids may be between 0.1 and 10.0 mg/mL, or between 0.5 and 5.0 mg/mL.

### D. Acyl Carnitines

Acyl carnitines are believed to enhance bioavailability, and in some embodiments are combined with a sugar ester of a fatty acid. Illustrative acyl carnitines include but are not limited to L-lauroyl carnitine and myristoyl carnitine. When used, concentration of acyl carnitine may be between 0.1 and 10.0 mg/mL, or between 0.5 and 5.0 mg/mL.

### E. Citrates

In some embodiments, citrate-type bioavailability enhancing agents selected from the group consisting of citric acid, citric acid salt and mixtures thereof are used in combination with one or more of the other enhancers discussed herein. Without intending to be bound by theory, it is believed that citrate-type enhancing agents may increase paracellular transport. In some embodiments, the concentration of all such citrate-type enhancing agents will be no lower than 5 mM and no higher than 50 mM, or in the range of 10-25 mM. Without intending to be bound by theory, it is believed that shelf stability may be undesirably reduced at higher citrate concentrations due to interaction of citrate with the active peptide at the amino terminus of the peptide, or at lysyl side chains.

### F. Other Embodiments

The above defined compositions may be applied in accordance with the methods to the nasal mucosa, e.g. either in drop or in spray form. The compositions of the present disclosure may of course also include additional ingredients, in particular components belonging to the class of conventional pharmaceutically applicable surfactants.

In some embodiments, the liquid pharmaceutical composition of the present methods contains a pharmaceutically acceptable diluent or carrier suitable for application to the nasal mucosa. Aqueous saline may be used for example.

The compositions of the present disclosure are formulated so as to permit administration via the nasal route. For this purpose they may also contain, e.g. minimum amounts of any additional ingredients or excipients desired, for example, additional preservatives or, e.g. ciliary stimulants such as caffeine.

Generally for nasal administration a mildly acid pH will be used. In some embodiments, the compositions of the present disclosure have a pH of from about 3.0 to 6.5.

The compositions of the present disclosure should also possess an appropriate isotonicity and viscosity. In some embodiments, they have an osmotic pressure of from about 260 to about 380 mOsm/liter. In some embodiments, the viscosity for the nasal spray is less than 0.98 cP.

Compositions in accordance with the present disclosure may also comprise a conventional surfactant, such as a non-ionic surfactant. When a surfactant is employed, the amount present in the compositions will vary depending on the particular surfactant chosen, the particular mode of administration (e.g. drop or spray) and the effect desired. In general, however, the amount present will be of the order of from about 0.1 mg/ml to about 10 mg/ml, about 0.5 mg/ml to 5 mg/ml, or about 1 mg/ml.

In some embodiments, a pharmaceutically acceptable preservative is included. Many are known in the art, and have been used in the past in connection with aqueous nasal pharmaceuticals. For example, benzyl alcohol or phenylethyl alcohol or a mixture thereof may be employed. In one embodiment, 0.2% phenylethyl alcohol and 0.5% benzyl alcohol are used in combination.

The amount of peptide to be administered, and hence the amount of active ingredient in the composition will, of course, depend on the particular peptide chosen, the condition to be treated, the desired frequency of administration and the effect desired.

The quantity of the total composition administered at each nasal application suitably comprises from about 0.05 to 0.15 ml, typically about 0.1 ml.

For the purposes of nasal administration, the compositions will be kept in a container provided with means enabling application of the contained composition to the nasal mucosa, e.g. put up in a nasal applicator device. Suitable applicators are known in the art and include those adapted for administration of liquid compositions to the nasal mucosa in drop or spray form. Because dosing should be as accurately controlled as possible, spray applicators for which the administered quantity is susceptible to precise regulation may be used. Suitable administrators include, e.g. atomizing devices, pump-atomizers and aerosol dispensers. In the latter case, the applicator will contain a composition in accordance with the methods together with a propellant medium suitable for use in a nasal applicator. The atomizing device will be provided with an appropriate spray adaptor allowing delivery of the contained composition to the nasal mucosa. Such devices are well known in the art.

The container, e.g., nasal applicator, may contain sufficient composition for a single nasal dosing or for the supply of several sequential dosages, e.g. over a period of days or weeks. Quantities of individual dosages supplied may be as hereinbefore defined.

In accordance with the present methods it has now been surprisingly found that pharmaceutical compositions can be obtained comprising aromatic-cationic peptide as an active ingredient which meet the high standards of stability and bioavailability required for nasal application and which are, for example, eminently suitable for use in multiple dose nasal spray applicators, i.e., applicators capable of delivering a series of individual dosages over, e.g., period of several days or weeks, by the use of citric acid or a salt thereof in concentrations ranging from about 10 to about 50 mM as a buffering agent.

Surprisingly, it has also been found that use of citric acid or a salt thereof at increasing concentrations confers beneficial advantages in relation to the nasal absorption characteristics of aromatic-cationic peptide containing compositions and hence enhance aromatic-cationic peptide bioavailability levels consequential to nasal application. In addition, it has also been found that the use of citric acid or a salt thereof in concentrations ranging from about 10 to about 50 mM increase the stability of aromatic-cationic peptide compositions while at the same time higher concentrations of citric acid or salt thereof do not have the same stabilizing effect.

The aromatic-cationic peptide for use in the present methods may be in free form or in pharmaceutically acceptable salt or complex form, e.g. in pharmaceutically acceptable acid addition salt form. Such salts and complexes are known and possess an equivalent degree of activity and tolerability to the free forms. Suitable acid addition salt forms for use in accordance with the methods include for example the hydrochlorides and acetates.

The above defined compositions may be applied in accordance with the methods to the nasal mucosa, e.g. either in drop or in spray form. As hereinafter described however, they may be applied in spray form, i.e., in the form of finely divided droplets.

The compositions of the present disclosure may of course also include additional ingredients, in particular components belonging to the class of conventional pharmaceutically applicable surfactants. In this connection it has in accordance with a further aspect of the present methods been found that the use of surface active agents generally in relation to the nasal application of aromatic-cationic peptides of the present technology, may increase absorption via the nasal mucosa and hence improve obtained bioavailability rates.

In some embodiments, the liquid pharmaceutical compositions of the present methods contain a pharmaceutically acceptable, a liquid diluent or carrier suitable for application to the nasal mucosa, such as aqueous saline.

The compositions of the disclosure are formulated so as to permit administration via the nasal route. For this purpose they may also contain, e.g. minimum amounts of any additional ingredients or excipients desired, for example, additional preservatives or, e.g. ciliary stimulants such as caffeine.

Generally for nasal administration a mildly acid pH will be used. In some embodiments the compositions have a pH of from about 3 to 5, about 3.5 to about 3.9 or about 3.7. Adjustment of the pH is achieved by addition of an appropriate acid, such as hydrochloric acid.

The compositions of the present disclosure should also possess an appropriate isotonicity and viscosity. In some embodiments they have an osmotic pressure of from about 260 to about 380 mOsm/liter. In some embodiments, the desired viscosity for the nasal spray is less than 0.98 cP. In one embodiment, the osmotic pressure is from 250 to 350 mOsm/liter.

Compositions in accordance with the present disclosure may also comprise a conventional surfactant, such as a non-ionic surfactant.

When a surfactant is employed, the amount present in the compositions will vary depending on the particular surfactant chosen, the particular mode of administration (e.g. drop or spray) and the effect desired. In general, however, the amount present will be of the order of from about 0.1 mg/ml to about 10 mg/ml, about 0.5 mg/ml to 5 mg/ml, or about 1 mg/ml.

The amount of aromatic-cationic peptide of the present technology to be administered in accordance with the present methods, and hence the amount of active ingredient in the composition will, of course, depend on the particular aromatic-cationic peptide chosen, the condition to be treated, the desired frequency of administration and the effect desired.

As indicated in the following examples, bioavailability for aromatic-cationic peptides of the present technology, as determined in terms of blood-plasma concentration following nasal administration in accordance with the teachings of the present methods has been found to be surprisingly high.

For nasal administration in accordance with the present methods, treatment will therefore suitably comprise administration of dosages at a frequency of from about once daily to about three times weekly. Dosages may be administered in a single application, i.e., treatment will comprise administration of single nasal dosages of aromatic-cationic peptide of the present technology. Alternatively such dosages may be split over a series of 2 to 4 applications taken at intervals during the day. The total composition quantity administered at each nasal application will vary according to the condition being treated, the particular peptide being administered, and the characteristics of the subject.

For the purposes of nasal administration, the compositions may be put up in a container provided with means enabling application of the contained composition to the nasal mucosa, e.g. put up in a nasal applicator device. Suitable applicators are known in the art and include those adapted for administration of liquid compositions to the nasal mucosa in drop or spray form. Since dosing with aromatic-cationic peptides of the present technology should be as accurately controlled as possible, spray applicators for which the administered quantity is susceptible to precise relation may be used. Suitable administrators include, e.g. atomizing devices, e.g. pump-atomizers and aerosol dispensers. In the latter case, the applicator will contain a composition in accordance with the methods together with a propellant medium suitable for use in a nasal applicator. The atomizing device will be provided with an appropriate spray adaptor allowing delivery of the contained composition to the nasal mucosa. Such devices are well known in the art.

The container, e.g. nasal applicator, may contain sufficient composition for a single nasal dosing or for the supply of several sequential dosages, e.g. over a period of days or weeks. Quantities of individual dosages supplied may be as hereinbefore defined. The stability of the compositions may be determined in conventional manner. As indicated herein below, the aromatic-cationic peptide content of the compositions will degrade less than 50% in 15 days at 50° C. as indicated by standard analytical tests.

### V. Methods of Treating

In some embodiments, treatments are administered as follows:

The aromatic-cationic peptides of the present technology and formulations thereof as provided herein are useful in treating any disease or condition that is associated with MPT. Such diseases and conditions include, but are not limited to, ischemia and/or reperfusion of a tissue or organ, hypoxia, diseases and conditions of the eye, myocardial infarction and any of a number of neurodegenerative diseases. Mammals in need of treatment or prevention of MPT are those mammals suffering from these diseases or conditions.

Ischemia in a tissue or organ of a mammal is a multifaceted pathological condition which is caused by oxygen deprivation (hypoxia) and/or glucose (e.g., substrate) deprivation. Oxygen and/or glucose deprivation in cells of a tissue or organ leads to a reduction or total loss of energy generating capacity and consequent loss of function of active ion transport across the cell membranes. Oxygen and/or glucose deprivation also leads to pathological changes in other cell membranes, including permeability transition in the mitochondrial membranes. In addition other molecules, such as apoptotic proteins normally compartmentalized within the mitochondria, may leak out into the cytoplasm and cause apoptotic cell death. Profound ischemia can lead to necrotic cell death.

Ischemia or hypoxia in a particular tissue or organ may be caused by a loss or severe reduction in blood supply to the tissue or organ. The loss or severe reduction in blood supply may, for example, be due to thromboembolic stroke, coronary atherosclerosis, or peripheral vascular disease. The tissue affected by ischemia or hypoxia is typically muscle, such as cardiac, skeletal, or smooth muscle.

The organ affected by ischemia or hypoxia may be any organ that is subject to ischemia or hypoxia. Examples of organs affected by ischemia or hypoxia include brain, heart, kidney, and prostate. For instance, cardiac muscle ischemia or hypoxia is commonly caused by atherosclerotic or thrombotic blockages which lead to the reduction or loss of oxygen delivery to the cardiac tissues by the cardiac arterial and capillary blood supply. Such cardiac ischemia or hypoxia may cause pain and necrosis of the affected cardiac muscle, and ultimately may lead to cardiac failure.

Ischemia or hypoxia in skeletal muscle or smooth muscle may arise from similar causes. For example, ischemia or hypoxia in intestinal smooth muscle or skeletal muscle of the limbs may also be caused by atherosclerotic or thrombotic blockages.

Reperfusion is the restoration of blood flow to any organ or tissue in which the flow of blood is decreased or blocked. For example, blood flow can be restored to any organ or tissue affected by ischemia or hypoxia. The restoration of blood flow (reperfusion) can occur by any method known to those in the art. For instance, reperfusion of ischemic cardiac tissues may arise from angioplasty, coronary artery bypass graft, or the use of thrombolytic drugs.

The methods of the present disclosure can also be used in the treatment or prophylaxis of neurodegenerative diseases associated with MPT. Neurodegenerative diseases associated with MPT include, for instance, Parkinson's disease, Alzheimer's disease, Huntington's disease and Amyotrophic Lateral Sclerosis (ALS, also known as Lou Gehrig's disease). The methods of the present disclosure can be used to delay the onset or slow the progression of these and other neurodegenerative diseases associated with MPT. The methods of the present disclosure are particularly useful in the treatment of humans suffering from the early stages of neurodegenerative diseases associated with MPT and in humans predisposed to these diseases.

The peptides useful in the present methods may also be used in preserving an organ of a mammal prior to transplantation. For example, a removed organ can be susceptible to MPT due to lack of blood flow. Therefore, the peptides can be used to prevent MPT in the removed organ.

The peptides may also be administered to a mammal taking a drug to treat a condition or disease. If a side effect of the drug includes MPT, mammals taking such drugs would greatly benefit from the oral formulations of aromatic-cationic peptides of the present technology disclosed herein.

The bioavailability achievable in accordance with the present technology permits oral delivery of aromatic-cationic peptides of the present technology into the blood at the above-identified concentration levels while using only 300-3000 micrograms of peptide per capsule, 300-1,200 micrograms, or between 300 and 600 micrograms.

It is optimal that a single tablet or capsule be used at each administration because a single dose of the product best provides simultaneous release of the aromatic-cationic peptide of the present technology, pH-lowering agent and absorption enhancers. This is highly desirable because the acid is best able to reduce undesirable proteolytic attack on the polypeptide when the acid is released in close time proximity to release of the polypeptide. Near simultaneous release is thus best achieved by administering all components of the present formulations as a single tablet or capsule. However, the present technology also includes, for example, dividing the required amount of acid and enhancers among two or more tablets or capsules which may be administered together such that they together provide the necessary amount of all ingredients. The term “Pharmaceutical composition,” as used herein includes a complete dosage appropriate to a particular administration to a human patient regardless of how it is subdivided so long as it is for substantially simultaneous administration.

Set forth below are a series of tables showing the predicted effect on bioavailability caused by varying certain parameters. Except with regard to prophetic human studies reported here, ingredient amounts may be varied from those described herein to account for differences between humans and the animals used in the animal models.

In some embodiments, treatments are administered as follows:

In some embodiments, a single capsule is used at each administration. In some embodiments, a single capsule best provides simultaneous release of the aromatic-cationic peptide, pH-lowering agent, and absorption enhancers. This is desirable because the acid is able to reduce undesirable proteolytic attack on the polypeptide when the acid is released in close time proximity to release of the polypeptide. Thus, in some embodiments, near simultaneous release is achieved by administering all components of the composition as a single pill or capsule. However, the present technology also includes, for example, dividing the required amount of acid and enhancers among two or more capsules which may be administered together such that they together provide the necessary amount of all ingredients. “Pharmaceutical composition,” as used herein includes a complete dosage appropriate to a particular administration to a human patient regardless of how it is subdivided so long as it is for substantially simultaneous administration.

For certain indications, it may be administered a first oral pharmaceutical composition in a capsule or tablet which does not contain a protective acid stable vehicle, such that the components will be relatively rapidly released in the stomach and thus be available for immediate pain relief, i.e., within about 10-20 minutes. Subsequently, additional capsules or tablets formulated according to the methods with a protective vehicle may then be administered, resulting in bioavailability in the intestine of the active ingredient after the longer time interval that is required for gastric emptying, i.e., typically around two hours.

In some embodiments, a sufficient amount of the aromatic-cationic peptide is included in the oral formulation of the composition to achieve a serum level (i.e., Cmax) of the aromatic-cationic peptide is from 200 μg/ml to 20 ng/ml, or from 200 μg/ml to 2 ng/ml. Dosage levels of the aromatic-cationic peptide for achieving the above serum levels may range from 100 μg to 10 mg, or from 100 μg to 1 mg. With respect to all of the dosages recommended herein, however, the attending clinician should monitor the subject's response and adjust the dosage accordingly. Moreover, except where otherwise stated, the dosage of the aromatic-cationic peptide of the present technology is identical for both therapeutic and prophylactic purposes. The dosage for each aromatic-cationic peptide discussed herein is the same, regardless of the disease being treated (or prevented). Furthermore, except where otherwise indicated, the terms “compound” and “composition”, and any associated molecular structure may include any possible stereoisomers thereof, in the form of a racemic mixture or in optically active form.

Except where otherwise noted, or where apparent from context, dosages herein refer to weight of aromatic-cationic peptide unaffected by pharmaceutical excipients, diluents, carriers or other ingredients, although such additional ingredients are desirably included.

### VI. Combination Therapy with an Aromatic-Cationic Peptide and Other Therapeutic Agents

In some embodiments, the aromatic-cationic peptides may be combined with one or more additional agents for the prevention or treatment of a disease or condition. For example, in some embodiments, an additional therapeutic agent is administered to a subject in combination with an aromatic-cationic peptide. In some embodiments, a synergistic therapeutic effect is produced. A “synergistic therapeutic effect” refers to a greater-than-additive therapeutic effect which is produced by a combination of two therapeutic agents (e.g., an aromatic-cationic peptide and another agent), and which exceeds that which would otherwise result from individual administration of either therapeutic agent alone. Therefore, lower doses of one or both of the therapeutic agents may be used in treating or preventing a disease or condition, resulting in increased therapeutic efficacy and decreased side-effects.

In any case, the multiple therapeutic agents (e.g., an aromatic-cationic peptide and another agent) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

In some embodiments, the other agent comprises an aromatic-cationic peptide. In some embodiments, the aromatic-cationic peptide is administered in conjunction with peptides for appetite suppression and weight control. In some embodiments, the peptide for appetite suppression and weight control is an calcitonin analog. In some embodiments, the peptide has the amino acid sequence Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Arg-Thr-Xaa-Xaa-Gly-Xaa-Xaa-Thr-Xaa, wherein amino acids 26, 27, 28, 29, and 31 can be any naturally occurring amino acid, and wherein amino acid 31 is optionally amidated.

### EXAMPLES

The formulations described herein are further illustrated by the following examples. The examples are intended to be illustrative only and are not to be construed as limiting in any way. The examples are intended to show trends relating to the formulations described herein and are not intended to limit the scope of composition or function of the formulations.

### Example 1

### Effects of pH on the Bioavailability of Aromatic-Cationic Peptides of the Present Technology

This example will demonstrate the effect of pH on the bioavailability of formulations comprising the aromatic-cationic peptides of the present technology, such as Phe-D-Arg-Phe-Lys-NH₂ and D-Arg-2′6′-Dmt-Lys-Phe-NH₂.

Female Wistar rats (250-275 g) (n=3 for each formulation) will be anesthetized with ketamine and xylazine prior to the insertion of a cannula in the carotid artery. The cannula will be fitted to a three way valve through which blood will be sampled and replaced with physiological saline. A midline incision will be made in the abdominal cavity and 0.5 ml of formulation will be injected directly into the exposed duodenum. The pH of the formulation will be adjusted by mixing citric acid and sodium citrate of equimolar concentrations. Blood (0.5 ml) will be collected before administration of the formulation and at 5, 15, 30, 60, and 120 minutes after the administration. Blood samples will be centrifuged for 10 minutes at 2600×g and the resulting plasma supernatant will be stored at −20° C. The concentration of aromatic-cationic peptide in plasma will be determined by reverse phase HPLC chromatography and/or mass spectroscopy (MS). One of skill in the art will understand that the aromatic-cationic peptides described herein may be analyzed by a number of HPLC methods, including reverse phase HPLC, such as those described in Aguilar, HPLC of Peptides and Proteins: Methods and Protocols, Humana Press, New Jersey (2004). Likewise, one of skill in the art will understand that the aromatic-cationic peptides described herein may be analyzed by a number of MS methods, such as those described in Sparkman, Mass Spectroscopy Desk Reference, Pittsburgh: Global View Pub (2000).

The absolute bioavailability or aromatic-cationic peptide (i.e., relative to an intravenous dose of aromatic-cationic peptide) will be calculated from the area under the curve obtained from plots of the plasma concentration of aromatic-cationic peptide as a function of time.

Anticipated trends in the effects of buffer pH on the bioavailability of aromatic-cationic peptide are shown in Table 4. It is anticipated that when the pH of the buffer is reduced from 5.0 (illustrative formulation I) to 4.0 (illustrative formulation II) the absolute bioavailability of aromatic-cationic peptide will increase as much as five-fold. It is expected that reduction of the buffer pH to 3.0 (illustrative formulation III) will increase the absolute bioavailability of the peptide as much as 32-fold compared to that achieved with buffer of pH 5.0. It is expected that reduction of the buffer pH to 2.0 (illustrative formulation IV) will result in very little additional increase in absolute bioavailability of the peptide. It is anticipated that a substantial increase in the absolute bioavailability of aromatic-cationic peptide will occur when the buffer pH is reduced from 5.0 to 3.0.

TABLE 4
Anticipated Effects of Buffer pH on the Bioavailability
of Aromatic-Cationic Peptide Absorbed From Rat Duodenum

| | | | Peak Plasma | Absolute |
|---|---|---|---|---|
| | | | Peptide | Bioavail- |
| | | | Concentration | ability |
| | Illustrative Formulation | pH | ng/ml* | Percent* |
| I | Aromatic-cationic peptide (0.1 mg) | 5 | 1x | 1x |
| | Citrate/Citric acid (77 mg) | | | |
| II | Aromatic-cationic peptide (0.1 mg) | 4 | 5x | 5x |
| | Citrate/Citric acid (77 mg) | | | |
| III | Aromatic-cationic peptide (0.1 mg) | 3 | 10x | 32x |
| | Citrate/Citric acid (77 mg) | | | |
| IV | Aromatic-cationic peptide (0.1 mg) | 2 | 12x | 34x |
| | Citrate Citric acid (77 mg) | | | |

*Relative to values obtained for formulation I

### Example 2

### Effects of Citric Acid on the Bioavailability of Aromatic-Cationic Peptides of the Present Technology

This example will demonstrate the effect of citric acid on the bioavailability of the aromatic-cationic peptides of the present technology, such as Phe-D-Arg-Phe-Lys-NH₂ and D-Arg-2′6′-Dmt-Lys-Phe-NH₂.

Formulations consisting of a fixed amount of taurodeoxycholic acid and two different amounts of citric acid will be prepared in a total volume of 0.5 ml. Mannitol will be included in the formulations as a marker to measure paracellular transport. The formulations will be administered to female Wistar rats as described in Example 1. Blood samples will be collected and bioavailability measured as described in Example 1.

Anticipated trends in the effect of citric acid on the bioavailability of aromatic-cationic peptide are shown in Table 5. It is anticipated that a relatively higher citric acid concentration will result in increased bioavailability of aromatic-cationic peptides of the present technology compared to a lower citric acid concentration. For example, illustrative formulation II is anticipated to increase the bioavailability of aromatic-cationic peptide by as much as 10-fold over that achieved with illustrative formulation I. In the presence of a fixed amount of taurodeoxycholic acid, the bioavailability of aromatic-cationic peptides of the present technology it is anticipated to increase when the amount of citric acid in the formulation is increased only 5 fold.

TABLE 5
Anticipated Effects of Citric Acid on the Bioavailability
of Aromatic-Cationic Peptide Absorbed From Rat Duodenum

| | | Peak Plasma | Absolute |
|---|---|---|---|
| | | Peptide | Bioavail- |
| | | Concentration | ability |
| | Ilustrative Formulation | ng/ml* | Percent* |
| I | Aromatic-cationic peptide (0.1 mg) | 1x | 1x |
| | Citric acid (9.6 mg) | | |
| | Taurodeoxycholic acid (5 mg) | | |
| | Mannitol (22 mg) | | |
| II | Aromatic-cationic peptide (0.1 mg) | 5x | 10x |
| | Citric acid (48 mg) | | |
| | Taurodeoxycholic acid (5 mg) | | |
| | Mannitol (22 mg) | | |

*Relative to values obtained for formulation I

### Example 3

### Effects of Absorption Enhancers on the Bioavailability of Aromatic-Cationic Peptides of the Present Technology

This example will demonstrate the effect of absorption enhancers on the bioavailability of the aromatic-cationic peptides of the present technology, such as Phe-D-Arg-Phe-Lys-NH₂ and D-Arg-2′6′-Dmt-Lys-Phe-NH₂.

Formulations consisting of citric acid, aromatic-cationic peptide, and various classes of enhancers will be prepared in a total volume of 0.5 ml. Mannitol will be included in formulation V as a marker to measure paracellular transport. The formulations will be administered to female Wistar rats as described in Example 1. Blood samples will be collected and bioavailability measured as described in Example 1. Anticipated trends in the effect of enhancers on the bioavailability of aromatic-cationic peptide are shown in Table 6. It is anticipated that formulations including an enhancer will result in increased bioavailability of aromatic-cationic peptide relative to formulations lacking an enhancer. The inclusion of a water soluble phospholipid (illustrative formulation VII) is expected to increase the bioavailability of aromatic-cationic peptide by as much as four-fold. The most effective enhancer is anticipated to be the sugar ester class (illustrative formulation V) in which the aromatic-cationic peptide bioavailability may be increased as much as eight-fold. The use of a mixture of bile acid and a cationic detergent (illustrative formulation III), a non-ionic detergent (illustrative formulation IV), or an acylcarnitine (illustrative formulation VI) are expected to increase the bioavailability of aromatic-cationic peptide as much as eight-fold compared to that achieved with illustrative formulation I. Variations in the bioavailability of aromatic-cationic peptide administered with various classes of enhancers are expected to be minor compared to variations observed when the peptide is formulated with citric acid only and no enhancer.

TABLE 6
Anticipated Effects of Enhancers in the Presence
of Citric Acid on the Absorption of Aromatic-
Cationic Peptide Absorbed From Rat Duodenum

| | | Peak Plasma | Absolute |
|---|---|---|---|
| | | Peptide | Bioavail- |
| | | Concentration | ability |
| | Illustrative Formulation | ng/ml* | Percent* |
| I | Aromatic-cationic peptide (0.1 mg) | 1x | 1x |
| | Citric acid (77 mg) | | |
| II | Aromatic-cationic peptide (0.1 mg) | 5x | 4x |
| | Citric acid (77 mg) | | |
| | Taurodeoxycholic acid (5 mg) | | |
| III | Aromatic-cationic peptide (0.1 mg) | 8x | 7x |
| | Citric acid (77 mg) | | |
| | Cetylpyridinium chloride (5 mg) | | |
| IV | Aromatic-cationic peptide (0.1 mg) | 3x | 5x |
| | Citric acid (48 mg) | | |
| | Tween -20 (5 mg) | | |
| V | Aromatic-cationic peptide (0.1 mg) | 8x | 8x |
| | Citric acid (48 mg)Sucrose ester-15 | | |
| | (5 mg) | | |
| | Mannitol (22 mg) | | |
| VI | Aromatic-cationic peptide (0.1 mg) | 8x | 8x |
| | Citric acid (48 mg) | | |
| | Lauroylcarnitine chloride (5 mg) | | |
| VII | Aromatic-cationic peptide (0.1 mg) | 4x | 4x |
| | Citric acid (48 mg) | | |
| | Diheptanoylphosphatidylcholine | | |
| | (5 mg) | | |

*Relative to values obtained for formulation I

### Example 4

### Effect of Lauroylcarnitine on the Bioavailability of Aromatic-Cationic Peptides of the Present Technology

This example will demonstrate the effect of lauroylcarnitine on the bioavailability of the aromatic-cationic peptides of the present technology, such as Phe-D-Arg-Phe-Lys-NH₂ and D-Arg-2′6′-Dmt-Lys-Phe-NH₂.

Formulations consisting of lauroylcarnitine, aromatic-cationic peptide of the present technology, and various other compounds will be prepared in a total volume of 0.5 ml. The formulations will be administered to female Wistar rats as described in Example 1. Blood samples will be collected and bioavailability measured as described in Example 1.

Anticipated trends in the effect of lauroylcarnitine on the bioavailability of aromatic-cationic peptide are shown in Table 7. It is anticipated that administration of aromatic-cationic peptide the absence of citric acid or any enhancer (illustrative formulation I) will result in reduced absolute bioavailability of peptide compared to formulations that include citric acid or an enhancer. It is anticipated that the inclusion of 5 mg lauroylcarnitine chloride (illustrative formulation II) will increase the bioavailability or aromatic-cationic peptide by approximately two-fold relative to illustrative formulation I. It is anticipated that the inclusion of lauroylcarnitine together with citric acid (illustrative formulation III), will increase the bioavailability of aromatic-cationic peptide by as much as 50-fold. It is anticipated that a five-fold reduction in the amount of lauroylcarnitine, but not citric acid (illustrative formulation IV), will not significantly reduce the bioavailability of aromatic-cationic peptide compared to that achieved with illustrative formulation III. It is expected that the inclusion of 5 mg diheptanoylphosphatidylcholine together with citric acid and lauroylcarnitine (illustrative formulation V) will increase the bioavailability of peptide by as much as 67-fold over that achieved with illustrative formulation I. The substitution of 25 mg bovine serum albumin for citric acid (illustrative formulation VI) is anticipated to increase the bioavailability of aromatic-cationic peptide compared to that achieved with illustrative formulation I (unformulated peptide), but to a lesser extent than illustrative formulations I-V. It is expected that these results will show the synergistic effects of pH-lowering agents (e.g. citric acid) and an enhancers (e.g. lauroylcarnitine) on the bioavailability of aromatic-cationic peptide.

TABLE 7
Anticipated Effect of Lauroylcarnitine in the Presence
of Additives on the Bioavailability of Aromatic-Cationic
Peptide Absorbed From Rat Duodenum

| | | Peak Plasma | Absolute |
|---|---|---|---|
| | | Peptide | Bioavail- |
| | | Concentration | ability |
| | Illustrative Formulation | ng/ml* | Percent* |
| I | Aromatic-cationic peptide (0.1 mg) | 1x | 1x |
| II | Aromatic-cationic peptide (0.1 mg) | 0.25x | 2x |
| | Lauroylcarnitine chloride (5 mg) | | |
| III | Aromatic-cationic peptide (0.1 mg) | 4x | 50x |
| | Lauroylcarnitine chloride (5 mg) | | |
| | Citric acid (48 mg) | | |
| IV | Aromatic-cationic peptide (0.1 mg) | 3x | 50x |
| | Lauroylcarnitine chloride (1 mg) | | |
| | Citric acid (48 mg) | | |
| V | Aromatic-cationic peptide (0.1 mg) | 5x | 67x |
| | Lauroylcarnitine chloride (5 mg) | | |
| | Diheptanoylphosphatidylcholine | | |
| | (5 mg) | | |
| | Bovine Serum Albumin (25 mg) | | |
| VI | Aromatic-cationic peptide (0.1 mg) | 0.5x | 4x |
| | Lauroylcarnitine chloride (5 mg) | | |
| | Bovine Serum Albumin (25 mg) | | |

*Relative to values obtained for formulation I

### Example 5

### Effect of Illustrative Formulations on the Absorption of Aromatic-Cationic Peptides of the Present Technology

This example will demonstrate the effect of illustrative formulations on absorption of the aromatic-cationic peptides of the present technology, such as Phe-D-Arg-Phe-Lys-NH₂ and D-Arg-2′6′-Dmt-Lys-Phe-NH₂.

Modified vascular access ports will be surgically implanted into the duodenum, ileum and colon of male beagle dogs. The septum/reservoir bodies of the ports will be implanted under the skin and will be used as sites for the administration of aromatic-cationic peptide formulations. Before and after the administration of aromatic-cationic peptide formulations into conscious dogs, the ports will be flushed with 2 ml of a mock formulation lacking aromatic-cationic peptide. Blood (2 ml) will be collected through angiocatheter tubes in the leg vein at 30, 15, and 0 minutes before administration of aromatic-cationic peptide, and at 5, 10, 20, 30, 40, 50, 60, and every 15 minutes thereafter for 2 hours after administration. Blood samples will be centrifuged for 10 minutes at 2600 g and the resulting plasma supernatant will be stored at −20° C. The concentration of aromatic-cationic peptide in plasma will be determined by a reverse-phase HPLC. The absolute bioavailability (i.e. relative to an intravenous dose of aromatic-cationic peptide) will be calculated from the areas under the curve obtained from plots of the plasma concentration as a function of time.

Anticipated trends in the effect of illustrative formulations on the bioavailability of aromatic-cationic peptide are shown in Table 8. It is anticipated that the absolute bioavailability of aromatic-cationic peptide administered alone (illustrative formulation I) will be low compared to formulations that include taurodeoxycholic acid and/or citric acid. It is anticipated that including citric acid in the formulation (illustrative formulation II) will increase the bioavailability of the peptide by as much as 25-fold. It is anticipated that further including taurodeoxycholic acid in the formulation (illustrative formulation III) will increase the bioavailability of the peptide by as much as 50-fold.

TABLE 8
Anticipated Effect of Illustrative Formulations on the Bioavailability
of Aromatic-Cationic Peptide Absorbed From Dog Duodenum

| | | Peak Plasma | Absolute |
|---|---|---|---|
| | | Peptide | Bioavail- |
| | | Concentration | ability |
| | Illustrative Formulation | ng/ml* | Percent* |
| I | Aromatic-Cationic Peptide (25 mg) | 1x | 1x |
| II | Aromatic-cationic peptide (25 mg) | 10x | 25x |
| | Citric Acid (192 mg) | | |
| III | Aromatic-cationic peptide (5 mg) | 13x | 50x |
| | Citric Acid (192 mg) | | |
| | Taurodeoxycholic Acid (20 mg) | | |

*Relative to values obtained for formulation I

### Example 6

### Effect of Citric Acid and Lauroylcarnitine on the Bioavailability of Vasopressin, Aromatic-Cationic Peptides of the Present Technology, and Insulin

This example will demonstrate the effect of citric acid lauroylcarnitine on the bioavailability of the aromatic-cationic peptides of the present technology, such as Phe-D-Arg-Phe-Lys-NH₂ and D-Arg-2′6′-Dmt-Lys-Phe-NH₂, vasopressin, and insulin.

Formulations consisting of either [Arg⁸]-vasopressin, aromatic-cationic peptide, or human insulin together with specified additives will be prepared in a total volume of 0.5 ml. The formulations will be administered to female Wistar rats as described in Example 1. Blood samples will be collected and bioavailability measured as described in Example 1.

Anticipated trends in the effect of lauroylcarnitine and citric acid on the bioavailability of vasopressin, aromatic-cationic peptide, and insulin are shown in Table 9. It is expected that the bioavailability of [Arg⁸]-vasopressin formulated with citric acid (illustrative formulation V-II) will be as much as 20-fold higher than that of unformulated [Arg⁸]-vasopressin (illustrative formulation V-I). It is expected that the bioavailability of aromatic-cationic peptide formulated with citric acid and lauroylcarnitine (illustrative formulation ACP-II) will be as much as 50-fold higher than that of unformulated peptide (illustrative formulation ACP-I). It is expected that the bioavailability of insulin formulated with citric acid and lauroylcarnitine (illustrative formulation HI-II) will be as much as 11-fold higher that that of unformulated insulin (illustrative formulation HI-I). These results are anticipated to demonstrate that the bioavailability of unformulated therapeutic peptides is substantially lower than peptides formulated with an organic acid, such as citric acid, and an enhancer, such as lauroylcarnitine.

TABLE 9
Anticipated Effect of Citric Acid and Lauroylcarnitine
on the Bioavailability of Vasopressin, Aromatic-
Cationic Peptide, and Insulin In Rats

| | | Peak Plasma | Absolute |
|---|---|---|---|
| | | Peptide | Bioavail- |
| | | Concentration | ability |
| | Illustrative Formulation | ng/ml* | Percent* |
| V-I | Yasopressin (1 mg) | 1x | 1x |
| V-II | Vasopressin (0.1 mg) | 40x | 20x |
| | Citric acid (48 mg) | | |
| ACP-I | Aromatic-cationic peptide (1 mg) | 1x | 1x |
| ACP-II | Aromatic-cationic peptide (0.1 mg) | 3x | 50x |
| | Citric acid (48 mg) | | |
| | Lauroylcarnitine (5 mg) | | |
| HI-I | Insulin (1 mg) | 1x | 1x |
| | Citric Acid (48 mg) | | |
| HI-II | Insulin (1 mg) | 33x | 11x |
| | Citric Acid (48 mg) | | |
| | Lauroylcarnitine (5 mg) | | |

Abbreviations:
V-I: [Arg⁸]-Vasopressin formulation I
V-II: [Arg⁸]-Vasopressin formulation II
ACP-I: Aromatic-cationic peptide formulation I
ACP-II: Aromatic-cationic peptide formulation II
HI-I: Human insulin formulation I
HI-II: Human insulin formulation II
*Relative to values obtained for formulation I in each group

### Example 7

### Effect of Enteric Coating on Absorption of Formulations Comprising Aromatic-Cationic Peptides of the Present Technology

This example will demonstrate the effect of enteric coating on absorption of illustrative formulations of the aromatic-cationic peptides of the present technology, such as Phe-D-Arg-Phe-Lys-NH₂ and D-Arg-2′6′-Dmt-Lys-Phe-NH₂.

Size 00 UPMC (hydroxypropylmethyl cellulose) capsules will each be filled with a powdered blend consisting of citric acid, lauroylcarnitine, and aromatic-cationic peptide. Half the capsules will be coated with an enteric coating solution of EUDRAGIT L30D-55 (a methacrylic acid co-polymer with methacrylic acid methyl ester, ROUM Tech Inc., Maidan, Mass.), and the remaining capsules will not be coated. The coating process will correspond to that taught in U.S. Pat. No. 6,086,918 at col. 11, line 50 to col. 12, line 11. The average capsule content for the enteric coated and non-enteric coated capsules is shown in Table 10.

Eight fasted dogs will be orally administered one uncoated capsule at week one. At week three, each subject will be orally administered one enteric-coated capsule. After each administration, samples of blood will taken at 15 minute intervals from an indwelling catheter for up to 4 hours. The blood samples will be centrifuged and the resulting plasma supernatants will be stored frozen at −20° C. The plasma samples will be analyzed for aromatic-cationic peptides of the present technology by reverse phase HPLC chromatography and/or mass spectroscopy (MS). The maximum plasma concentration of aromatic-cationic peptides of the present technology will be normalized to a 1 mg dose.

Anticipated trends in the effect of enteric coating on the bioavailability of aromatic-cationic peptides of the present technology is shown in Table 10. It is anticipated that aromatic-cationic peptides of the present technology will be detected in plasma from dogs orally administered enteric coated as well as uncoated capsules. It is anticipated that maximal plasma concentrations will be about three-fold higher following administration of enteric-coated capsules as compared to non-coated capsules. It is anticipated that maximum plasma concentrations will be achieved within 30 minutes after administration of uncoated capsules, and 90 minutes after administration of enteric-coated capsules.

It is anticipated that these results will demonstrate that a therapeutically effective amount of an aromatic-cationic peptide is absorbed from non-coated capsules at a faster rate than from enteric-coated capsules, and that higher plasma concentrations will be achieved with coated capsules than with non-coated capsules. Faster rates of absorption may be advantageous, especially in the case of peptides wherein speed is more important than overall bioavailability (e.g., inhibition of MPT). There can also be an advantage in production efficiency when the enteric coating step is not required.

TABLE 10
Anticipated Effect of Enteric Coating on Absorption of Aromatic-
cationic peptides of the present technology in Dogs

| | | Illustrative | Illustrative | | |
|---|---|---|---|---|---|
| | Illustrative | Lauroyl- | Aromatic- | | |
| Enteric | Citric acid | Carnitine | cationic | Cₘₐₓ | Tₘₐₓ |
| coat | (mg) | (mg) | peptide (mg) | (pg/ml)* | (min)** |
| No | 643 | 66 | 13.07 | 1x | approx. 30 |
| Yes | 632 | 65 | 12.84 | 3x | approx. 90 |

*Relative to values obtained for uncoated capsules
**Time when maximum plasma concentration detected

### Example 8

### Effects of Illustrative Formulations on Absorption of Aromatic-Cationic Peptide from Non-Enteric Coated Capsules

This example will demonstrate the effect of illustrative formulations on absorption of the aromatic-cationic peptides of the present technology, such as Phe-D-Arg-Phe-Lys-NH₂ and D-Arg-2′6′-Dmt-Lys-Phe-NH₂.

Size 00 UPMC (hydroxypropylmethyl cellulose) capsules will each be filled with a powdered blend consisting of the indicated amount of citric acid, lauroylcarnitine, sucrose and aromatic-cationic peptide. The average capsule content for the capsules is shown in Table 11. Each week, eight fasted dogs will each be orally administered one uncoated capsule. After each administration, samples of blood will be taken at 15 minute intervals from an indwelling catheter for up to 4 hours. The blood samples will be centrifuged and the resulting plasma supernatants will be stored frozen at −20° C. The plasma samples will be subsequently analyzed for aromatic-cationic peptide by reverse phase HPLC chromatography and/or mass spectroscopy (MS).

Anticipated trends in the effect of illustrative formulations on absorption of aromatic-cationic peptide from non-enteric coated capsules are shown in Table 11. It is anticipated that administration of aromatic-cationic peptide alone (illustrative formulation I) will result in a relatively lower plasma concentration than formulations including citric acid and/or lauroylcarnitine. It is expected that administration of peptide together with lauroylcarnitine (illustrative formulation II), citric acid (illustrative formulation III), or both (illustrative formulation IV) will result in as much as 50-fold, 230-fold, and 270-fold higher plasma concentrations than achieved with unformulated peptide, respectively. These results are anticipated to demonstrate the importance of including both an acid and an absorption enhancer in aromatic-cationic peptide formulations.

TABLE 11
Anticipated Effects of Illustrative Formulations on Absorption of
Aromatic-Cationic Peptide From Non-enteric Coated Capsules in Dogs

| | | | | Aromatic- | | |
|---|---|---|---|---|---|---|
| | | Lauroyl- | | cationic | | |
| Illustrative | Citric acid | Carnitine | Sucrose | peptide | Cₘₐₓ | Tₘₐₓ |
| Formulation | (mg) | (mg) | (mg) | (mg) | (pg/ml)* | (min)** |
| I | 0 | 0 | 805 | 5.31 | 1x | approx. 30 |
| II | 0 | 70 | 712 | 6.17 | 50x | approx. 30 |
| III | 824 | 0 | 0 | 5.31 | 230x | approx. 30 |
| IV | 679 | 67 | 0 | 5.37 | 270x | approx. 30 |

*Relative to values obtained for unformulated aromatic-cationic peptide
**Time when maximum plasma aromatic-cationic peptide concentration detected.

### Example 9

### Effects of Illustrative Formulations Absorption of the Aromatic-Cationic Peptides of the Present Technology, Such as Phe-D-Arg-Phe-Lys-NH₂ and D-Arg-2′6′-Dmt-Lys-Phe-NH₂

Size 00 UPMC capsules will each be filled with a powdered blend consisting of at least 500 mg citric acid, 50 mg lauroylcarnitine and 1.0 mg of the aromatic-cationic peptide Phe-D-Arg-Phe-Lys-NH₂ or D-Arg-2′6′-Dmt-Lys-Phe-NH₂, or a pharmaceutically acceptable salt thereof, such as acetate or trifluoroacetate salt. Each week, eight fasted dogs will be orally administered one uncoated capsule. After each administration, samples of blood will be taken at 15 minute intervals from an indwelling catheter for up to 4 hours. The blood samples will be centrifuged and the resulting plasma supernatants will be stored frozen at −20° C. The plasma samples will subsequently be analyzed for Phe-D-Arg-Phe-Lys-NH₂ or D-Arg-2′6′-Dmt-Lys-Phe-NH₂ as described in Example 7.

Anticipated trends in the effect of illustrative formulations on absorption of the aromatic-cationic peptides of the present technology Phe-D-Arg-Phe-Lys-NH₂ or D-Arg-2′6′-Dmt-Lys-Phe-NH₂ from non-enteric coated capsules are shown in Table 12. It is anticipated that administration of the peptide alone (illustrative formulation I) will result in a relatively lower plasma concentration than formulations including citric acid and/or lauroylcarnitine. It is expected that administration of peptides together with lauroylcarnitine (illustrative formulation II), citric acid (illustrative formulation III), or both (illustrative formulation IV) will result in as much as 50-fold, 230-fold, and 270-fold higher plasma concentrations than achieved with unformulated peptide, respectively. These results are anticipated to demonstrate the importance of including both an acid and an absorption enhancer in Phe-D-Arg-Phe-Lys-NH₂ and D-Arg-2′6′-Dmt-Lys-Phe-NH₂ peptide formulations.

TABLE 12
Anticipated Effects of Illustrative Formulations on Absorption of
Phe-D-Arg-Phe-Lys-NH₂ and D-Arg-2′6′-Dmt-Lys-Phe-NH₂ From
Non-enteric Coated Capsules in Dogs:

| | | | | Aromatic- | | |
|---|---|---|---|---|---|---|
| | | Lauroyl- | | cationic- | | |
| Illustrative | Citric acid | Carnitine | Sucrose | peptides | Cₘₐₓ | Tₘₐₓ |
| Formulation | (mg) | (mg) | (mg) | (mg)*** | (pg/ml)* | (min)** |
| I | 0 | 0 | 805 | 5.31 | 1x | approx. 30 |
| II | 0 | 70 | 712 | 6.17 | 50x | approx. 30 |
| III | 824 | 0 | 0 | 5.31 | 230x | approx. 30 |
| IV | 679 | 67 | 0 | 5.37 | 270x | approx. 30 |

*Relative to values obtained for unformulated peptide
**Time when maximum plasma aromatic-cationic peptide concentration detected
***The aromatic-cationic peptides of the present technology Phe-D-Arg-Phe-Lys-NH₂ or D-Arg-2′6′-Dmt-Lys-Phe-NH₂, or pharmaceutically acceptable salts thereof, such as acetate or trifluoroacetate salt.

Although the present method has been described in relation to particular embodiments thereof, many other variations and modifications and other uses will become apparent to those skilled in the art. The present method therefore is not limited by the specific technology herein, but only by the claims.

### Example 10

### Effects of Enteric Coating on the Bioavailability of Aromatic Cationic Peptides of the Present Technology

This example will demonstrate the effect of illustrative formulations on absorption of the aromatic-cationic peptides of the present technology, such as Phe-D-Arg-Phe-Lys-NH₂ and D-Arg-2′6′-Dmt-Lys-Phe-NH₂.

Administration of aromatic-cationic peptide in the oral formulation described herein provides unexpected improvements in bioavailability of the subject peptide.

With regard to a first series of tests, i.e., on rats, the improved effect will be demonstrated by comparing the curves for formulated aromatic-cationic peptide vs. unformulated aromatic-cationic peptide. Six anesthetized rats will be given 0.7 mL aromatic-cationic peptide (1.6 mg/mL) with a syringe through a 27 gauge needle into the duodenum. This injection procedure will be followed due to the technical difficulty inherent in preparing capsules which can be swallowed by small animals the size of a rat. The intraduodanal injection, therefore, will mimic the release of the components of an enteric-coated capsule formulation which would pass through the esophagus and stomach and release its contents in the duodenum. Three of the rats will be given unformulated aromatic-cationic peptide in which there are no additional components (i.e., other than the aromatic-cationic peptide), while the other three rats will be given formulated aromatic-cationic peptide which include, in addition to the aromatic-cationic peptide, 0.5M citric acid and lauroyl carnitine (10 mg/ml). Samples of blood will be taken from the carotid artery through an indwelling catheter before and 5, 15, 30, 60 and 120 minutes after the administration of the respective formulations (i.e., formulated and unformulated).

The blood samples will be centrifuged and the resulting plasma supernatants will be stored frozen at −20° C. The plasma samples will be subsequently analyzed for aromatic-cationic peptide by high-performance liquid chromatography (HPLC) through a 50×4.6 mm polysulfoethyl-aspartamid-e column with a mobile phase of 15.4 mM potassium phosphate (pH 3), 210 mM sodium chloride, and 25% acrylonitrile at a flow rate of 1.5 mL/min. Peptide will be detected with an ultraviolet (UV) detector set at a wavelength of 210 nm. The results are expected to show that aromatic-cationic peptide is virtually undetectable in rats given unformulated aromatic-cationic peptide, whereas as much as 8 μg/mL of aromatic-cationic peptide is predicted to be detectable in rats given aromatic-cationic peptide formulated in citric acid and lauroyl carnitine. These results are expected to demonstrate that formulating aromatic-cationic peptide in an oral formulation according to the present methods increases the Cmax and AUC compared to the unformulated peptide.

A second series of tests will be carried out, as noted above, using beagle dogs. The improved bioavailability of orally administered aromatic-cationic peptide will be demonstrated in this second series of tests by comparing the curves for (1) non-enteric coated salmon calcitonin (sCT) and (2) non-enteric coated aromatic-cationic peptide with the curves for (3) enteric coated sCT and (4) enteric coated aromatic-cationic peptide. In the experiments, size 00 HPLC capsules will be filled with 758 mg of a powdered blend consisting of citric acid (643 mg), lauroyl carnitine (66 mg), talc (33 mg), salmon calcitonin (sCT) (13 mg) and aromatic-cationic peptide (2.4 mg). Half of the capsules will be coated with an enteric coating solution of L30D-55, while the remaining 50% of the capsules will not be coated. Four fasted dogs will be each given 1 uncoated capsule, and 2 weeks later they will be each given an enteric coated capsule. After administration of each capsule, samples of blood will be taken at 15 minute intervals from an indwelling catheter for up to 4 hours. The blood samples will be centrifuged and the resulting plasma supernatants will be stored frozen at −20° C. The plasma samples will be subsequently analyzed for sCT by a direct ELISA, and for aromatic-cationic peptide by HPLC-mass spectrometry performed as set forth in Wan, H. and Desiderio, D., Quantitation of dmt-DALDA in ovine plasma by on-line liquid chromatography/quadrapole time-of-flight mass spectrometry, Rapid Communications in Mass Spectrometry, 2003; 17, 538-546, the contents of which are incorporated herein by reference.

The results will be summarized as plasma peptide concentration normalized to a 1 mg dose as a function of time relative to the average Tmax, (i.e., the time at which the maximum amount of peptide is detected). The results are expected to indicate that both peptides, i.e., sCT and aromatic-cationic peptide, are detected in dogs given uncoated or enteric coated capsules. It is expected that nearly three times as much aromatic-cationic peptide as sCT will be detected in dogs given uncoated capsules; whereas, nearly equal amounts of both peptides will be detected in dogs given enteric coated capsules. It is expected that nearly four times as much aromatic-cationic peptide will be detected in the plasma of dogs given enteric coated capsules than those given non-coated capsules. It is expected that nearly eight times as much sCT will be detected in the plasma of dogs given enteric coated capsules than non-coated capsules. The maximum concentration of aromatic-cationic peptide and sCT in dogs given uncoated capsules is expected to be seen 30 minutes after their administration, whereas the maximum concentration of these materials when given in coated capsules is expected to be seen 105 minutes after their administration, due to the additional time necessary for the oral formulation to pass through the stomach while remaining protected from the proteolytic enzymes therein. These results are expected to demonstrate that coating the capsules with an enteric polymer such that the capsule does not release its contents until reaching the small intestine, significantly enhances peptide absorption.

The Cmax and AUC values for both sCT and aromatic-cationic peptide are expected to be significantly enhanced when the peptides are administered in enteric coated capsules versus in non enteric-coated capsules. The Cmax of enteric coated aromatic-cationic peptide is expected to be 4-fold higher than that of non enteric coated aromatic-cationic peptide. The bioavailability of both enteric coated and non-coated aromatic-cationic peptide is expected to be better than that of sCT. It would be expected that the bioavailability of a molecule such as aromatic-cationic peptide, which is positively charged and hydrophilic, would be extremely poor. The data is expected to indicate that when this peptide is administered in combination with the ingredients of the present composition, either with or without an enteric coating, the bioavailability is increased to the point where it is superior to that of sCT, a molecule that has previously been shown to be highly bioavailable when formulated according to the present methods.

### Example 11

### Effect of OmPA-MT3 on the Absorption of Aromatic-Cationic Peptides of the Present Technology from Rat Duodenum

The following example will demonstrate the effect of OmPA-MT3 on the absorption of aromatic-cationic peptides of the present technology, such as Phe-D-Arg-Phe-Lys-NH₂ and D-Arg-2′6′-Dmt-Lys-Phe-NH₂ from rat duodenum. Female Sprague-Dawley rats (250-275 g) (n=4 for each peptide) are anesthetized with ketamine and xylazine prior to the insertion of a cannula in the carotid artery. The cannula is fitted to a three way valve through which blood is sampled and replaced with physiological saline containing heparin. A midline incision is made in the abdominal cavity, and 0.45 mL of either aromatic-cationic peptide (10 mg/mL) or OmpA-MT3-aromatic-cationinc peptide (10 mg/mL) in 0.5M citric acid is injected directly into the duodenum. Blood (0.5 ml) is collected before and at 5, 15, 30, 45 and 60 minutes after administration of the peptides. The blood is centrifuged, and the concentration (.+−. SEM [standard error of the mean]) of aromatic-cationic peptide or OmpA-MT3-aromatic-cationinc peptide in the plasma supernatant is determined by a competitive enzyme immunoassay (EIA). Peak plasma concentration (Cmax) is determined by inspection. The absolute bioavailability of each peptide (relative to an intravenous dose of aromatic-cationic-peptide is calculated from plots of the plasma concentration of each peptide as a function to time.

It is predicted that the maximum concentration of aromatic-cationic-peptide in the blood will be reached between 30 and 60 minutes after their administration. The Cmax of OmpA-MT3-aromatic-cationinc peptide is expected to be more than 25 fold greater than that of aromatic-cationic peptide. The bioavailability of OmpA-MT3-aromatic-cationinc peptide is expected to be more than 20 times greater than that of aromatic-cationic peptide. These results will indicate that attaching OmpA-MT3 to aromatic-cationic peptide significantly enhances peptide absorption through the intestinal wall.

### Example 12

### Effect of the HIV TAT Protein Transduction Domain as an MT on the Absorption of Aromatic-Cationic Peptides of the Present Technology from Dog Duodenum

Two formulations are used to test the efficacy of MT3-aromatic-cationic peptide fusion. The first formulation (F1) is prepared by blending 13 g citric acid, 1.3 g lauroylcarnitine, 0.65 g talc and 0.03 g aromatic-cationic peptide with a mortar and pestle. The other formulation (F2) is prepared by blending the same mixture except that s aromatic-cationic peptide is replaced with an equivalent amount of MT3-aromatic-cationic peptide. Both blends are used to fill size 00 gelatin capsules, and the capsules are coated with Eudragit L30D-55. The resulting enteric-coated capsules contain approximately 1 to 2 mg of either aromatic-cationic peptide (F1) or MT3-aromatic-cationic peptide (F2) per capsule. Fasted dogs (n=8) are administered F1 by mouth and blood samples are collected in heparinized tubes at t=−10 min, 0 min, and every 15 min thereafter for 240 minutes. The blood samples are centrifuged, and the resulting plasma stored at −20° C. for further analysis. After a 1 week washout period, the same dogs are given F2 by mouth, and the same protocol is followed.

The amount of aromatic-cationic peptide in plasma samples of dogs given either of the two formulations is HPLC using methods known in the art. Both formulations are expected to produce measurable amounts of aromatic-cationic peptide in the blood, the maximum concentration of aromatic-cationic peptide in the blood of dogs given F1 is expected to be in the range of 0.5 to 6.0 ng/ml, whereas the maximum concentration of aromatic-cationic peptide in dogs given F2 is expected to be at least 1 to 12 ng/ml.

The bioavailability of aromatic-cationic peptide in dogs given F1 is expected to be approximately 1%, whereas the bioavailability of aromatic-cationic peptide in dogs given F2 is expected to be at least 1.2%. The in vivo cleavage of MT from aromatic-cationic peptide in dogs given F2 is proven by applying samples of plasma from dogs given F1 and F2 to an HPLC column and collecting the effluent in plastic tubes. The solvent in the tubes is removed under vacuum and analyzed for the presence of aromatic-cationic peptide by HPLC. The in vivo cleavage of MT3-aromatic-cationic peptide is established by showing that the retention time of aromatic-cationic peptide in the plasma from dogs given F2 is the same as the retention time of aromatic-cationic peptide in the plasma of dogs given F1.

### Example 13

### Effect of Enhancer on Intranasal Absorption of Aromatic-Cationic Peptides of the Present Technology

The following example will demonstrate the effect of enhancers of the intranasal absorption of aromatic-cationic peptides of the present technology, such as Phe-D-Arg-Phe-Lys-NH₂ and D-Arg-2′6′-Dmt-Lys-Phe-NH₂. Female Sprague-Dawley rats, weighing between 225 and 250 g, are used in these studies. Rats are fasted overnight prior to administration of the test substance, but are allowed free access to water. Rats are anesthetized with a combination of ketamine and xylazine and a cannula is inserted into the carotid artery for blood sampling. The volume of each blood sample collected is 0.5 mL.

A 20 μL dose is administered by touching the left nostril with the disposable tip of an Eppendorf micropipette and gently applying pressure to the plunger of the pipette. Blood samples are collected prior to dosing and at 10, 20, 40, 60 and 120 minutes after the administration of aromatic-cationic peptide (1-2 mg/mL) in 0.85% sodium chloride.

The concentration of aromatic-cationic peptide in plasma is determined using an ELISA. Briefly, the assay consists of incubating rat samples in 96 well ELISA plates that are coated with rabbit antibody to aromatic-cationic peptide After incubating and washing the plates, goat antibody to aromatic-cationic peptide is added to the plates. Bound antibody is detected with rabbit anti-goat IgG-horse-radish conjugate and 3,3′,5,5′-Tetramethylbenzidine peroxide substrate after washing off unbound goat antibody.

Rats are given intranasal aromatic-cationic peptide (1-2 mg/mL) in 16 mM sodium phosphate/8 mm citric acid (pH 4.8) containing 0.85% sodium chloride and the indicated final concentration of enhancer.

It is predicted that the replacement of 0.1% Tween 80 with 0.2% LLC will increase the mean Cmax of aromatic-cationic peptide at least 3 fold, and that increasing the amount of LLC to 0.5% will not further increase the mean Cmax of aromatic-cationic peptide. It is expected that replacing 0.1% Tween 80 with 0.2% SL will increase the Cmax of aromatic-cationic peptide 2 fold. It is expected that adding up to 0.5% SL will not further increase the mean Cmax; however, inclusion of 1% SL in the formulation, is expected to increase the mean Cmax increased by nearly 4 fold.

Rats are given intranasal aromatic-cationic peptide (1 mg/mL) in 20 mm citric acid/sodium citrate (pH 3.8) containing 0.85% sodium chloride and the indicated final concentration of enhancer. Sodium oleate is added to the formulation prior to the addition of citrate buffer.

It is expected that the addition of sucrose laurate to the formulation will increase the Cmax of aromatic-cationic peptide nearly 2 fold and the inclusion sodium oleate increased the Cmax of aromatic-cationic peptide 2.6 fold. At pH 3.8 sodium oleate exists as oleic acid, which is insoluble in water. To overcome this problem, oleic acid is added to the formulation as sodium oleate prior to the addition of citrate buffer.

### Example 14

### Methods of Administering Aromatic-Cationic Peptides of the Present Technology and Measurement of Plasma Concentration

The following example will demonstrate methods of administering aromatic-cationic peptides of the present technology, such as Phe-D-Arg-Phe-Lys-NH₂ and D-Arg-2′6′-Dmt-Lys-Phe-NH₂, and measurement of plasma concentrations. Female Wistar rats, weighing between 225 and 250 g are anesthetized with a combination of ketamine and xyalzine, and a cannula is inserted into the carotid artery. The cannula is fitted to a three-way valve through which blood is sampled and replaced with physiological saline containing heparin. Formulated aromatic-cationic peptide (5 μg per 25 μl) is administered intranasally through a micropipette tip inserted 8 mm into the rat's nostril. For single-dose studies, 5 μg of aromatic-cationic peptide is administered. In multiple dose studies, aromatic-cationic peptide is administered four times in a volume of 25 μl each at 0, 30, 60 and 90 minutes for a total dose of 20 μg.

In single-dose studies, blood samples are collected prior to dosing and at 5, 15, 30, 60 and 120 minutes after dosing. In multiple-dose studies, blood samples are collected prior to dosing and at 30, 60, 90, 120 and 150 minutes after the administration of the first dose. Blood samples are always collected immediately before the administration of any additional doses.

Each sample (0.5 ml) of blood is collected into a heparinized 1 ml syringes and then transferred to chilled 1.5 ml polypropylene tubes containing 10 μl of heparin (500 U per ml). The tubes are centrifuged at approximately 3000 rpm for 20 minutes at 2-8° C. and the plasma supernatant is transferred to microcentrifuge tubes that are stored at −20° C. The concentration of aromatic-cationic peptide in plasma is determined by HPLC using methods known in the art.

The values of Cmax are determined by inspection and the values for bioavailability (relative to an intravenous injection) are calculated from the areas under the curve that is obtained from plots of plasma aromatic-cationic peptide concentration as a function of time.

The following example will demonstrate the effect of the concentration of citric acid on the bioavailability and plasma concentration of nasally administered aromatic-cationic peptides of the present technology, such as Phe-D-Arg-Phe-Lys-NH₂ and D-Arg-2′6′-Dmt-Lys-Phe-NH₂. Rats are administered intranasally as described previously 20 μl of aromatic-cationic peptide (200 μg/ml) in 0.85% sodium chloride, 0.1% Tween 80, 0.2% phenylethyl alcohol, 0.5% benzyl alcohol and varying amounts of citric acid adjusted to pH 3.7 at t=0, 20, 60 and 90 minutes. Samples of blood are taken prior to the administration of aromatic-cationic peptide at these time points as well as at t=120 and 150 minutes. The resulting plasma samples are analyzed for aromatic-cationic peptide by HPLC. Maximum aromatic-cationic peptide levels are expected to be detected at t=120 minutes. It is expected that the bioavailability and peak concentration of aromatic-cationic peptide will be a function of the concentration of citric acid in the formulation. It is expected that relatively higher concentrations of citric acid in the formulations will result in higher levels of bioavailability and peak serum concentration as compared to control formulations lacking citric acid.

The following study will demonstrate the effect of different preservatives on the plasma concentration of nasally administered aromatic-cationic peptides of the present technology, such as Phe-D-Arg-Phe-Lys-NH₂ and D-Arg-2′6′-Dmt-Lys-Phe-NH₂. Rats are administered intranasally as described previously 20 μl of aromatic-cationic peptide (200 μg/ml) in 0.85% sodium chloride, 0.1% Tween 80 and a combination preservatives of either 0.2% phenylethyl alcohol and 0.5% benzyl alcohol or 0.27% methyl parabens and 0.04% proply parabens at t=0, 30, 60 and 90 minutes. It is expected that the bioavailability and peak concentration of aromatic-cationic peptide will not significantly affected by the addition of the different preservatives.

The following study will demonstrate the effect of the concentration of citric acid on the stability of aromatic-cationic peptide stored for varying periods at a temperature of 50° C. Nasal formulations containing aromatic-cationic peptide (200 μg/ml), 0.25% phenylethyl alcohol, 0.5% benzyl alcohol and 0.1% Tween 80 are adjusted to pH 3.7 with either HCl or the indicated amount of buffered citric acid. The formulations are stored at 50° C. in sealed glass containers for the indicated amount of time and analyzed for aromatic-cationic peptide by high performance liquid chromatography. It is expected that in the absence of citric acid, the amount aromatic-cationic peptide in the formulation will decrease steadily between 0 and 9 days, but that in presence of citric acid (10-50 mM) the rate of disappearance of aromatic-cationic peptide will decrease significantly. It is further expected that as the concentration of citric acid is further increased, the rate of aromatic-cationic peptide disappearance from vials stored at 50° C. will increase in proportion to the amount of buffered citric acid in the formulation.

Although the present method has been described in relation to particular embodiments thereof, many other variations and modifications and other uses will become apparent to those skilled in the art. The present method therefore is not limited by the specific disclosure herein, but only by the claims.

1. A finished pharmaceutical product adapted for oral delivery of an aromatic-cationic peptide, the product comprising:
(a) a therapeutically effective amount of the aromatic-cationic peptide;
(b) at least one pharmaceutically acceptable pH-lowering agent; and
(c) at least one absorption enhancer effective to promote bioavailability of the active agent,
herein the pH-lowering agent is present in the finished pharmaceutical product in a quantity which, if the product were added to 10 milliliters of 0.1M aqueous sodium bicarbonate solution, would be sufficient to lower the pH of the solution to no higher than 5.5, and wherein an outer surface of the product is substantially free of an acid-resistant protective vehicle.

2. The finished pharmaceutical product of claim 1, wherein the pH-lowering agent is present in a quantity which, if the product were added to 10 milliliters of 0.1M sodium bicarbonate solution, would be sufficient to lower the pH of the solution to no higher than 3.5.

3. The finished pharmaceutical product of claim 1, wherein the absorption enhancer is an absorbable or biodegradable surface active agent.

4. The finished pharmaceutical product of claim 3, wherein the surface active agent is selected from the group consisting of acylcarnitines, phospholipids, bile acids and sucrose esters.

5. The finished pharmaceutical product of claim 1, wherein the absorption enhancer is a surface active agent selected from the group consisting of
(a) an anionic agent that is a cholesterol derivative,
(b) a mixture of a negative charge neutralizer and an anionic surface active agent,
(c) non-ionic surface active agents, and
(d) cationic surface active agents.

6. The finished pharmaceutical product of claim 1, further comprising an amount of a second peptide that is not a physiologically active peptide effective to enhance bioavailability of the aromatic-cationic peptide.

7. The finished pharmaceutical product of claim 1, wherein at least one pH-lowering agent has a solubility in water of at least 30 grams per 100 milliliters of water at room temperature.

8. The finished pharmaceutical product of claim 1, wherein the product comprises granules containing a pharmaceutical binder and, uniformly dispersed in the binder, the pH-lowering agent, the absorption enhancer and the aromatic-cationic peptide.

9. The finished pharmaceutical product of claim 1, wherein the product comprises a lamination having a first layer comprising the at least one pharmaceutically acceptable pH-lowering agent and a second layer comprising the therapeutically effective amount of the aromatic-cationic peptide; the product further comprising the at least one absorption enhancer effective to promote bioavailability of the active agent, wherein the first and second layers are united with each other, but the at least one pH-lowering agent and the aromatic-cationic peptide are substantially separated within the lamination such that less than about 0.1% of the aromatic-cationic peptide contacts the pH-lowering agent to prevent substantial mixing between the first layer material and the second layer material and thus to avoid interaction in the lamination between the pH-lowering agent and the aromatic-cationic peptide.

10. The finished pharmaceutical product of claim 1, wherein the pH-lowering agent is selected from the group consisting of citric acid, tartaric acid and an acid salt of an amino acid.

11. The finished pharmaceutical product of claim 1, wherein the pH-lowering agent is selected from the group consisting of dicarboxylic acids and tricarboxylic acids.

12. (canceled)

13. The finished pharmaceutical product of claim 1, wherein the aromatic-cationic peptide comprises the amino acid sequence Phe-D-Arg-Phe-Lys-NH₂ or D-Arg-2′6′-Dmt-Lys-Phe-NH₂.

14. (canceled)

15. The finished pharmaceutical product of claim 1, wherein the aromatic-cationic peptide is selected from the group consisting of:
Phe-Arg-D-His-Asp
Met-Tyr-D-Lys-Phe-Arg
Phe-D-Arg-His
Tyr-D-Arg-Phe-Lys-NH₂
2′6′-Dmt-D-Arg-Phe-Lys-NH₂
2′6′-Dmt-D-Arg-Phe Orn-NH₂
2′6′-Dmt-D-Cit-Phe Lys-NH₂
Phe-D-Arg-2′6′-Dmt-Lys-NH₂
2′6′-Dmt-D-Arg-Phe-Ahp-NH₂
H-Phe-D-Arg-Phe-Lys-Cys-NH₂
2′6′-Dmp-D-Arg-2′6′-Dmt-Lys-NH₂
2′6′-Dmp-D-Arg-Phe-Lys-NH₂
Tyr-Arg-Phe-Lys-Glu-His-Trp-D-Arg
Lys-Gln-Tyr-D-Arg-Phe-Trp
D-Arg-2′6′-Dmt-Lys-Trp-NH₂
D-Arg-Trp-Lys-Trp-NH₂
D-Arg-2′6′-Dmt-Lys-Phe-Met-NH₂
D-Arg-2′6′-Dmt-Lys(N^{α}Me)-Phe-NH₂
D-Arg-2′6′-Dmt-Lys-Phe(NMe)—NH₂
D-Arg-2′6′-Dmt-Lys(N^{α}Me)-Phe(NMe)—NH₂
D-Arg(N^{α}Me)-2′6′-Dmt(NMe)-Lys(N^{α}Me)-Phe(NMe)—NH₂
D-Arg-2′6′-Dmt-Lys-Phe-Lys-Trp-NH₂
D-Arg-2′6′-Dmt-Lys-2′6′-Dmt-Lys-Trp-NH₂
D-Arg-2′6′-Dmt-Lys-Phe-Lys-Met-NH₂
D-Arg-2′6′-Dmt-Lys-2′6′-Dmt-Lys-Met-NH₂
D-Arg-2′6′-Dmt-Lys-Phe-Sar-Gly-Cys-NH₂
D-Arg-Ψ[CH₂—NH]2′6′-Dmt-Lys-Phe-NH₂
D-Arg-2′6′-Dmt-Ψ[CH₂—NH]Lys-Phe-NH₂
D-Arg-2′6′-Dmt-LysΨ[CH₂—NH]Phe-NH₂
D-Arg-2′6′-Dmt-Ψ[CH₂—NH]Lys-Ψ[CH₂—NH]Phe-NH₂
Lys-D-Arg-Tyr-NH₂
D-Tyr-Trp-Lys-NH₂
Trp-D-Lys-Tyr-Arg-NH₂
Tyr-His-D-Gly-Met
Tyr-D-Arg-Phe-Lys-Glu-NH₂
Met-Tyr-D-Arg-Phe-Arg-NH₂
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr-D-Phe-Glu-D-Asp-D-Asp-D-His-D-Lys-Arg-Trp-NH₂
Ala-D-Phe-D-Arg-Tyr-Lys-D-Trp-His-D-Tyr-Gly-Phe
Tyr-D-His-Phe-D-Arg-Asp-Lys-D-Arg-His-Trp-D-His-Phe
Phe-Phe-D-Tyr-Arg-Glu-Asp-D-Lys-Arg-D-Arg-His-Phe-NH₂
Phe-Tyr-Lys-D-Arg-Trp-His-D-Lys-D-Lys-Glu-Arg-D-Tyr-Thr
Tyr-Asp-D-Lys-Tyr-Phe-D-Lys-D-Arg-Phe-Pro-D-Tyr-His-Lys
Glu-Arg-D-Lys-Tyr-D-Val-Phe-D-His-Trp-Arg-D-Gly-Tyr-Arg-D-Met-NH₂
Arg-D-Leu-D-Tyr-Phe-Lys-Glu-D-Lys-Arg-D-Trp-Lys-D-Phe-Tyr-D-Arg-Gly
D-Glu-Asp-Lys-D-Arg-D-His-Phe-Phe-D-Val-Tyr-Arg-Tyr-D-Tyr-Arg-His-Phe-NH₂
Asp-Arg-D-Phe-Cys-Phe-D-Arg-D-Lys-Tyr-Arg-D-Tyr-Trp-D-His-Tyr-D-Phe-Lys-Phe
His-Tyr-D-Arg-Trp-Lys-Phe-D-Asp-Ala-Arg-Cys-D-Tyr-His-Phe-D-Lys-Tyr-His-Ser-NH₂
Gly-Ala-Lys-Phe-D-Lys-Glu-Arg-Tyr-His-D-Arg-D-Arg-Asp-Tyr-Trp-D-His-Trp-His-D-Lys-Asp
Thr-Tyr-Arg-D-Lys-Trp-Tyr-Glu-Asp-D-Lys-D-Arg-His-Phe-D-Tyr-Gly-Val-Ile-D-His-Arg-
Tyr-Lys-NH₂

16. A method for enhancing the bioavailability of a therapeutic aromatic-cationic peptide delivered orally in a subject in need of such enhancement, the method comprising selectively releasing the aromatic-cationic peptide, together with at least one pH-lowering agent and at least one absorption enhancer, into the subject's alimentary canal from a finished pharmaceutical product adapted for delivery of the aromatic-cationic peptide, wherein an outer surface of the product is substantially free of an acid resistant protective vehicle, wherein the pharmaceutical product is released into the alimentary canal in a quantity which, if added to 10 milliliters of 0.1M aqueous sodium bicarbonate solution, would be sufficient to lower pH of the solution to no higher than 5.5.

17. The method of claim 16, wherein the therapeutic aromatic-cationic peptide, the at least one pH-lowering agent and the at least one absorption enhancer are released from the finished pharmaceutical product more rapidly than from a corresponding pharmaceutical composition comprising an acid resistant protective vehicle.

18. (canceled)

19. The method of claim 16, wherein the pH-lowering agent is present in a quantity which, if all ingredients were added to 10 milliliters of 0.1M aqueous sodium bicarbonate solution, would be sufficient to lower the pH of the solution to no higher than 3.5.

20. The method of claim 16, wherein the absorption enhancer is selected from the group consisting of a cationic surfactant and an anionic surfactant that is a cholesterol derivative.

21-22. (canceled)

23. The method of claim 16, wherein the aromatic-cationic peptide comprises the amino acid sequence Phe-D-Arg-Phe-Lys-NH₂ or D-Arg-2′6′-Dmt-Lys-Phe-NH₂.

24. (canceled)

25. The method of claim 16, wherein the aromatic-cationic peptide is selected from the group consisting of:
Phe-Arg-D-His-Asp
Met-Tyr-D-Lys-Phe-Arg
Phe-D-Arg-His
Tyr-D-Arg-Phe-Lys-NH₂
2′6′-Dmt-D-Arg-Phe-Lys-NH₂
2′6′-Dmt-D-Arg-Phe Orn-NH₂
2′6′-Dmt-D-Cit-Phe Lys-NH₂
Phe-D-Arg-2′6′-Dmt-Lys-NH₂
2′6′-Dmt-D-Arg-Phe-Ahp-NH₂
H-Phe-D-Arg-Phe-Lys-Cys-NH₂
2′6′-Dmp-D-Arg-2′6′-Dmt-Lys-NH₂
2′6′-Dmp-D-Arg-Phe-Lys-NH₂
Tyr-Arg-Phe-Lys-Glu-His-Trp-D-Arg
Lys-Gln-Tyr-D-Arg-Phe-Trp
D-Arg-2′6′-Dmt-Lys-Trp-NH₂
D-Arg-Trp-Lys-Trp-NH₂
D-Arg-2′6′-Dmt-Lys-Phe-Met-NH₂
D-Arg-2′6′-Dmt-Lys(N^{α}Me)-Phe-NH₂
D-Arg-2′6′-Dmt-Lys-Phe(NMe)—NH₂
D-Arg-2′6′-Dmt-Lys(N^{α}Me)-Phe(NMe)—NH₂
D-Arg(N^{α}Me)-2′6′-Dmt(NMe)-Lys(N^{α}Me)-Phe(NMe)—NH₂
D-Arg-2′6′-Dmt-Lys-Phe-Lys-Trp-NH₂
D-Arg-2′6′-Dmt-Lys-2′6′-Dmt-Lys-Trp-NH₂
D-Arg-2′6′-Dmt-Lys-Phe-Lys-Met-NH₂
D-Arg-2′6′-Dmt-Lys-2′6′-Dmt-Lys-Met-NH₂
D-Arg-2′6′-Dmt-Lys-Phe-Sar-Gly-Cys-NH₂
D-Arg-Ψ[CH₂—NH]2′6′-Dmt-Lys-Phe-NH₂
D-Arg-2′6′-Dmt-Ψ[CH₂—NH]Lys-Phe-NH₂
D-Arg-2′6′-Dmt-LysΨ[CH₂—NH]Phe-NH₂
D-Arg-2′6′-Dmt-Ψ[CH₂—NH]Lys-Ψ[CH₂—NH]Phe-NH₂
Lys-D-Arg-Tyr-NH₂
D-Tyr-Trp-Lys-NH₂
Trp-D-Lys-Tyr-Arg-NH₂
Tyr-His-D-Gly-Met
Tyr-D-Arg-Phe-Lys-Glu-NH₂
Met-Tyr-D-Arg-Phe-Arg-NH₂
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr-D-Phe-Glu-D-Asp-D-Asp-D-His-D-Lys-Arg-Trp-NH₂
Ala-D-Phe-D-Arg-Tyr-Lys-D-Trp-His-D-Tyr-Gly-Phe
Tyr-D-His-Phe-D-Arg-Asp-Lys-D-Arg-His-Trp-D-His-Phe
Phe-Phe-D-Tyr-Arg-Glu-Asp-D-Lys-Arg-D-Arg-His-Phe-NH₂
Phe-Tyr-Lys-D-Arg-Trp-His-D-Lys-D-Lys-Glu-Arg-D-Tyr-Thr
Tyr-Asp-D-Lys-Tyr-Phe-D-Lys-D-Arg-Phe-Pro-D-Tyr-His-Lys
Glu-Arg-D-Lys-Tyr-D-Val-Phe-D-His-Trp-Arg-D-Gly-Tyr-Arg-D-Met-NH₂
Arg-D-Leu-D-Tyr-Phe-Lys-Glu-D-Lys-Arg-D-Trp-Lys-D-Phe-Tyr-D-Arg-Gly
D-Glu-Asp-Lys-D-Arg-D-His-Phe-Phe-D-Val-Tyr-Arg-Tyr-D-Tyr-Arg-His-Phe-NH₂
Asp-Arg-D-Phe-Cys-Phe-D-Arg-D-Lys-Tyr-Arg-D-Tyr-Trp-D-His-Tyr-D-Phe-Lys-Phe
His-Tyr-D-Arg-Trp-Lys-Phe-D-Asp-Ala-Arg-Cys-D-Tyr-His-Phe-D-Lys-Tyr-His-Ser-NH₂
Gly-Ala-Lys-Phe-D-Lys-Glu-Arg-Tyr-His-D-Arg-D-Arg-Asp-Tyr-Trp-D-His-Trp-His-D-Lys-Asp
Thr-Tyr-Arg-D-Lys-Trp-Tyr-Glu-Asp-D-Lys-D-Arg-His-Phe-D-Tyr-Gly-Val-Ile-D-His-Arg-
Tyr-Lys-NH₂

26. (canceled)

27. A method for enhancing the bioavailability of an orally delivered physiologically aromatic-cationic peptide comprising: (A) linking the aromatic-cationic peptide to a membrane translocator that possesses the capability of being at least partially cleaved in vivo by an enzyme; and (B) selectively releasing the aromatic-cationic peptide linked to the membrane translocator, together with at least one pH-lowering agent and/or protease inhibitor into a patient's intestine following passage of the aromatic-cationic peptide, pH-lowering agent and/or protease inhibitor through the patient's mouth and stomach under protection of an acid resistant protective vehicle which substantially prevents contact between stomach proteases and the aromatic-cationic peptide.

28-39. (canceled)
